SEQUENCE LISTING (18 sequences)

SEQ ID NO: 1 - DNA, 3000 bp, Escherichia coli
tttgttcttcccgtgatgcagacataattctatgaaagcataaattaaaaacgcacagaagcgtagaacgttatgtctggtttataaaatgaaccttcaattttattttttatgaaaacagcatttcatttttatggtttcgtttataccgatggtttatgtggaaattgtcgaagagagcagatttgcgcaacgctgggatcagtcttaaaaagtaaaaaaatatatttgcttgaacgattcaccgtttttttcatccggttaaatatgcaaagataaatgcgcagaaatgtgtttctcaaaccgttcatttatcacaaaaggattgttcgatgtccaacaatggctcgtcaccgctggtgctttggtataaccaactcggcatgaatgatgtagacagggttgggggcaaaaatgcctccctgggtgaaatgattactaatctttccggaatgggtgtttccgttccgaatggtttcgccacaaccgccgacgcgtttaaccagtttctggaccaaagcggcgtaaaccagcgcatttatgaactgctggataaaacggatattgacgatgttactcagcttgcgaaagcgggcgcgcaaatccgccagtggattatcgacactcccttccagcctgagctggaaaacgccatccgcgaagcctatgcacagctttccgccgatgacgaaaacgcctcttttgcggtgcgctcctccgccaccgcagaagatatgccggacgcttcttttgccggtcagcaggaaaccttcctcaacgttcagggttttgacgccgttctcgtggcagtgaaacatgtatttgcttctctgtttaacgatcgcgccatctcttatcgtgtgcaccagggttacgatcaccgtggtgtggcgctctccgccggtgttcaacggatggtgcgctctgacctcgcatcatctggcgtgatgttctccattgataccgaatccggctttgaccaggtggtgtttatcacttccgcatggggccttggtgagatggtcgtgcagggtgcggttaacccggatgagttttacgtgcataaaccgacactggcggcgaatcgcccggctatcgtgcgccgcaccatggggtcgaaaaaaatccgcatggtttacgcgccgacccaggagcacggcaagcaggttaaaatcgaagacgtaccgcaggaacagcgtgacatcttctcgctgaccaacgaagaagtgcaggaactggcaaaacaggccgtacaaattgagaaacactacggtcgcccgatggatattgagtgggcgaaagatggccacaccggtaaactgttcattgtgcaggcgcgtccggaaaccgtgcgctcacgcggtcaggtcatggagcgttatacgctgcattcacagggtaagattatcgccgaaggccgtgctatcggtcatcgcatcggtgcgggtccggtgaaagtcatccatgacatcagcgaaatgaaccgcatcgaacctggcgacgtgctggttactgacatgaccgacccggactgggaaccgatcatgaagaaagcatctgccatcgtcaccaaccgtggcggtcgtacctgtcacgcggcgatcatcgctcgtgaactgggcattccggcggtagtgggctgtggagatgcaacagaacggatgaaagacggtgagaacgtcactgtttcttgtgccgaaggtgataccggttacgtctatgcggagttgctggaatttagcgtgaaaagctccagcgtagaaacgatgccggatctgccgttgaaagtgatgatgaacgtcggtaacccggaccgtgctttcgacttcgcctgcctaccgaacgaaggcgtgggccttgcgcgtctggaatttatcatcaaccgtatgattggcgtccacccacgcgcactgcttgagtttgacgatcaggaaccgcagttgcaaaacgaaatccgcgagatgatgaaaggttttgattctccgcgtgaattttacgttggtcgtctgactgaagggatcgcgacgctgggtgccgcgttttatccgaagcgcgtcattgtccgtctctctgattttaaatcgaacgaatatgccaacctggtcggtggtgagcgttacgagccagatgaagagaacccgatgctcggcttccgtggcgcgggccgctatgtttccgacagcttccgcgactgtttcgcgctggagtgtgaagcagtgaaacgtgtgcgcaacgacatgggactgaccaacgttgagatcatgatcccgttcgtgcgtaccgtagatcaggcgaaagcggtggttgaagaactggcgcgtcaggggctgaaacgtggcgagaacgggctgaaaatcatcatgatgtgtgaaatcccgtccaacgccttgctggccgagcagttcctcgaatatttcgacggcttctcaattggctcaaacgatatgacgcagctggcgctcggtctggaccgtgactccggcgtggtgtctgaattgttcgatgagcgcaacgatgcggtgaaagcactgctgtcgatggctatccgtgccgcgaagaaacagggcaaatatgtcgggatttgcggtcagggtccgtccgaccacgaagactttgccgcatggttgatggaagaggggatcgatagcctgtctctgaacccggacaccgtggtgcaaacctggttaagcctggctgaactgaagaaataaaataaatccccggcggcgtttagtcgccggggttatgtgatccccgaagatgaaacttattcaatctcttcacagacatcctgcgttaaacgccgcataatatcttttcttaacaaaaacttttgtattttacctgaggtagttcgcggtagtttttcgattaccacgatatgttcaggatatttatattttgcgacccgtttacggctaaaaaaagccactacctcttccagcgataatgaatgatgcggcgctttcagcacgacataagcgcatgatcgttcaccta

SEQ ID NO: 2 - protein, 792 aa, Escherichia coli
MSNNGSSPLVLWYNQLGMNDVDRVGGKNASLGEMITNLSGMGVSVPNGFATTADAFNQFLDQSGVNQRIYELLDKTDIDDVTQLAKAGAQIRQWIIDTPFQPELENAIREAYAQLSADDENASFAVRSSATAEDMPDASFAGQQETFLNVQGFDAVLVAVKHVFASLFNDRAISYRVHQGYDHRGVALSAGVQRMVRSDLASSGVMFSIDTESGFDQVVFITSAWGLGEMVVQGAVNPDEFYVHKPTLAANRPAIVRRTMGSKKIRMVYAPTQEHGKQVKIEDVPQEQRDIFSLTNEEVQELAKQAVQIEKHYGRPMDIEWAKDGHTGKLFIVQARPETVRSRGQVMERYTLHSQGKIIAEGRAIGHRIGAGPVKVIHDISEMNRIEPGDVLVTDMTDPDWEPIMKKASAIVTNRGGRTCHAAIIARELGIPAVVGCGDATERMKDGENVTVSCAEGDTGYVYAELLEFSVKSSSVETMPDLPLKVMMNVGNPDRAFDFACLPNEGVGLARLEFIINRMIGVHPRALLEFDDQEPQLQNEIREMMKGFDSPREFYVGRLTEGIATLGAAFYPKRVIVRLSDFKSNEYANLVGGERYEPDEENPMLGFRGAGRYVSDSFRDCFALECEAVKRVRNDMGLTNVEIMIPFVRTVDQAKAVVEELARQGLKRGENGLKIIMMCEIPSNALLAEQFLEYFDGFSIGSNDMTQLALGLDRDSGVVSELFDERNDAVKALLSMAIRAAKKQGKYVGICGQGPSDHEDFAAWLMEEGIDSLSLNPDTVVQTWLSLAELKK

SEQ ID NO: 3 - DNA, 3062 bp, Pantoea ananatis
aagtcgtgcctcgggcagggaagtcgtaaaaacaagtgagataatataagcatgccttcaggggcaagtgtagtctggggcatcgtttccctctcgcttttgcatcatgacgtcacctgttcactcttttttgcgccattaaaaccgcctcaggtgacagacaacgcctgtgtttgttgcgcaacaaaaacgccgcataacaggcagtaaaaaaaataatgagctaattatcagcgtattagcaaaaaataagggccttgcgcaacgctgaaaaaaaccaggcgcaattctgaaactgtgtctgagaatctgctttaacgattcaacagatcgtgtttaatcagacgttatgccaggctggatttgttaatagcaggattttccatgtgcccattcattaataaaggatcagttcaatgtccaataaaggcgaacagccgttagtactttggtacaaccagcttggcatgcatgatgttgatcgggtgggaggcaaaaatgcttctctgggtgagatgattaccaatttgtcatctctgggcgtgtccgttcccaacggttttgcgaccacctcctgggcctttaatcagtttcttgagcagagcggtctgaaccagcgtatttatgctttgctggatgacaccaatattgatgatgtggatcaactggcgaaggcgggtaagcagatacgccagtgggtagtcgaaaccccgttccagcctgaactggaagccgcgattctttcggcctatgagcagctctctgccgacgatgctgaggcatccttcgccgtgcgctcatccgccaccgccgaagatatgcctgacgcgtcctttgccggccagcaggaaactttcctgaacgtgcagggcatcgagtcagtcatggtggcagtgaaacacgtttatgcctcactgtttaacgatcgcgccatctcctatcgcgtccatcagggttacgaccatcgcggcgtggcgctgtcggcggggattcagcgcatggtgcgatccgatctggcgtcctcaggggtcatgttcaccattgataccgaatcgggctttgatcaggtggtgttcattaccgccgcgttgggcctgggtgaaatggtggtacagggcgcggtgaacccggacgagttttatgtgcacaagccgacgctggccgcgaaacgtcccgctatcgttcgccgcaacatgggctcgaaaaaggtgcggatggtttacgccgactcgcgtgaacatggcgagcaggtgcgcatcgaagatgtggcggaagccgagcgcgatcgtttctgtctgagcgatgccgaagtggaggcgttggcccatcaggcggtactgattgagcaacactacaagcgcccgatggatattgagtgggccaaagacggccacaccgggaagttatttattgtccaggcgcgccctgaaaccgtgcgatccaacggtcaggtcatggagcgttacagcctgcagggacagggcaaggtggtgattgaaggccgtgcgattggtcaccgcatcggcgccggtaccgtcaaggtcattcacgatatcagcgaaatgaaccgcatcgaaaaaggtgacgtgctggtgaccgatatgaccgatcccgactgggaaccgatcatgaaaaaagcctccgccattgtgacgaaccgcggcggccgtacctgccatgccgcgattatcgcgcgtgagctgggcatcccggcggtggttggctgtggcgatgccaccgagcggctgaaagagggacataccgttaccgtatcctgtgctgagggcgataccggctatgtctacgatgaactgctcgacttcgacgtcaccagttcgcaggttgataccatgccggacctgccgcttaaaatcatgatgaacgtgggcaacccggatcgcgcgttcgatttcgcctgtttgccaaacgaaggggttggtctggcgcggctggaatttattatcaaccgcatgatcggcgtgcatccaaaagcgttgctggagttcgatcagcaaacccctgaactgcagaagcagattcgtcagatgatgaaaggttttgacgatccggttgagttttacatcgcccgtctgaccgaagggatcgcgacgctgggcgcggcgttcgcgccgaagcgcgttattgtccgcctgtcggattttaaaacgaacgaatatgccaatctggtgggtggagaacgttacgaacccgaagaggaaaacccgatgctggggttccgtggcgcaggccgctatgtctccgagagtttccgcgactgtttcgcgctggagtgcgaagcggtgaagcgcgtacgcaacgagatggggctgaccaacgtggaaatcatggtgccctttgtgcgtacggtcgatcaggctcaggcggtggtagaggaattgggtcgccagggactgaagcgcggcgagaacgggctcaaggtcattatgatgtgtgaaatcccctcgaatgccctgctggccgagcagtttttacaacactttgacggtttctccatcggctctaatgatatgactcagctggcgctggggctggatcgcgactccggcgtggtatctgccttgtttgatgagcgtaatgaggcggtcaaagcgctgctgtccatggcgattcaggcggcgaaaaaacagggcaaatacgtaggaatatgcggtcaggggccgtcagatcatcaggatttcgcggcctggttgatggagcagggcatcgacagtctgtctctgaatccggacaccgtggtggaaacctggttaagcctggcggcgctaaacaaacccgcctgaggtgatggataacctccgatgccagaaggccagcgcaagctggcttttttttttgtctttttgacaggcaaaaaaaagcccatcacaggggatgggcaaagactacacacagcaattctttacgctactcaggggaaattagggcgtttagaaatcagtaacttgatttccaacataggaaacatggtattcacacggctcgctggcgtctttaagaatcctcttattagtttaaaggaaataatattttgacggctaggg

SEQ ID NO: 4 - protein, 794 aa, Pantoea ananatis
MSNKGEQPLVLWYNQLGMHDVDRVGGKNASLGEMITNLSSLGVSVPNGFATTSWAFNQFLEQSGLNQRIYALLDDTNIDDVDQLAKAGKQIRQWVVETPFQPELEAAILSAYEQLSADDAEASFAVRSSATAEDMPDASFAGQQETFLNVQGIESVMVAVKHVYASLFNDRAISYRVHQGYDHRGVALSAGIQRMVRSDLASSGVMFTIDTESGFDQVVFITAALGLGEMVVQGAVNPDEFYVHKPTLAAKRPAIVRRNMGSKKVRMVYADSREHGEQVRIEDVAEAERDRFCLSDAEVEALAHQAVLIEQHYKRPMDIEWAKDGHTGKLFIVQARPETVRSNGQVMERYSLQGQGKVVIEGRAIGHRIGAGTVKVIHDISEMNRIEKGDVLVTDMTDPDWEPIMKKASAIVTNRGGRTCHAAIIARELGIPAVVGCGDATERLKEGHTVTVSCAEGDTGYVYDELLDFDVTSSQVDTMPDLPLKIMMNVGNPDRAFDFACLPNEGVGLARLEFIINRMIGVHPKALLEFDQQTPELQKQIRQMMKGFDDPVEFYIARLTEGIATLGAAFAPKRVIVRLSDFKTNEYANLVGGERYEPEEENPMLGFRGAGRYVSESFRDCFALECEAVKRVRNEMGLTNVEIMVPFVRTVDQAQAVVEELGRQGLKRGENGLKVIMMCEIPSNALLAEQFLQHFDGFSIGSNDMTQLALGLDRDSGVVSALFDERNEAVKALLSMAIQAAKKQGKYVGICGQGPSDHQDFAAWLMEQGIDSLSLNPDTVVETWLSLAALNKPA

SEQ ID NO: 5 - DNA, 2379 bp, Escherichia coli
atgtccaacaatggctcgtcaccgctggtgctttggtataaccaactcggcatgaatgatgtagacagggttgggggcaaaaatgcctccctgggtgaaatgattactaatctttccggaatgggtgtttccgttccgaatggtttcgccacaaccgccgacgcgtttaaccagtttctggaccaaagcggcgtaaaccagcgcatttatgaactgctggataaaacggatattgacgatgttactcagcttgcgaaagcgggcgcgcaaatccgccagtggattatcgacactcccttccagcctgagctggaaaacgccatccgcgaagcctatgcacagctttccgccgatgacgaaaacgcctcttttgcgatgcgctcctccgccaccgcagaagatatgccggacgcttcttttgccggtcagcaggaaaccttcctcaacgttcagggttttgacgccgttctcgtggcagtgaaacatgtatttgcttctctgtttaacgatcgcgccatctcttatcgtgtgcaccagggttacgatcaccgtggtgtggcgctctccgccggtgttcaacggatggtgcgctctgacctcgcatcatctggcgtgatgttctccattgataccgaatccggctttgaccaggtggtgtttatcacttccgcatggggccttggtgagatggtcgtgcagggtgcggttaacccggatgagttttacgtgcataaaccgacactggcggcgaatcgcccggctatcgtgcgccgcaccatggggtcgaaaaaaatccgcatggtttacgcgccgacccaggagcacggcaagcaggttaaaatcgaagacgtaccgcaggaacagcgtgacatcttctcgctgaccaacgaagaagtgcaggaactggcaaaacaggccgtacaaattgagaaacactacggtcgcccgatggatattgagtgggcgaaagatggccacaccggtaaactgttcattgtgcaggcgcgtccggaaaccgtgcgctcacgcggtcaggtcatggagcgttatacgctgcattcacagggtaagattatcgccgaaggccgtgctatcggtcatcgcatcggtgcgggtccggtgaaagtcatccatgacatcagcgaaatgaaccgcatcgaacctggcgacgtgctggttactgacatgaccgacccggactgggaaccgatcatgaagaaagcatctgccatcgtcaccaaccgtggcggtcgtacctgtcacgcggcgatcatcgctcatgaactgggcattccggcgatagtgggctgtggagatgcaacagaacggatgaaagacggtgagaacgtcactgtttcttgtgccgaaggtgataccggttacgtctatgcggagttgctggaatttagcgtgaaaagctccagcgtagaaacgatgccggatctgccgttgaaagtgatgatgaacgtcggtaacccggaccgtgctttcgacttcgcctgcctaccgaacgaaggcgtgggccttgcgcgtctggaatttatcatcaaccgtatgattggcgtccacccacgcgcactgcttgagtttgacgatcaggaaccgcagttgcaaaacgaaatccgcgagatgatgaaaggttttgattctccgcgtgaattttacgttggtcgtctgactgaagggatcgcgacgctgggtgccgcgttttatccgaagcgcgtcattgtccgtctctctgattttaaatcgaacgaatatgccaacctggtcggtggtgagcgttacgagccagatgaagagaacccgatgctcggcttccgtggcgcgggccgctatgtttccgacagcttccgcgactgtttcgcgctggagtgtgaagcagtgaaacgtgtgcgcaacgacatgggactgaccaacgttgagatcatgatcccgttcgtgcgtaccgtagatcaggcgaaagcggtggttgaagaactggcgcgtcaggggctgaaacgtggcgagaacgggctgaaaatcatcatgatgtgtgaaatcccgtccaacgccttgctggccgagcagttcctcgaatatttcgacggcttctcaattggctcaaacgatatgacgcagctggcgctcggtctggaccgtgactccggcgtggtgtctgaattgttcgatgagcgcaacgatgcggtgaaagcactgctgtcgatggctatccgtgccgcgaagaaacagggcaaatatgtcgggatttgcggtcagggtccgtccgaccacgaagactttgccgcatggttgatggaagaggggatcgatagcctgtctctgaacccggacaccgtggtgcaaacctggttaagcctggctgaactgaagaaataa

SEQ ID NO: 6 - protein, 792 aa, Escherichia coli
MSNNGSSPLVLWYNQLGMNDVDRVGGKNASLGEMITNLSGMGVSVPNGFATTADAFNQFLDQSGVNQRIYELLDKTDIDDVTQLAKAGAQIRQWIIDTPFQPELENAIREAYAQLSADDENASFAMRSSATAEDMPDASFAGQQETFLNVQGFDAVLVAVKHVFASLFNDRAISYRVHQGYDHRGVALSAGVQRMVRSDLASSGVMFSIDTESGFDQVVFITSAWGLGEMVVQGAVNPDEFYVHKPTLAANRPAIVRRTMGSKKIRMVYAPTQEHGKQVKIEDVPQEQRDIFSLTNEEVQELAKQAVQIEKHYGRPMDIEWAKDGHTGKLFIVQARPETVRSRGQVMERYTLHSQGKIIAEGRAIGHRIGAGPVKVIHDISEMNRIEPGDVLVTDMTDPDWEPIMKKASAIVTNRGGRTCHAAIIAHELGIPAIVGCGDATERMKDGENVTVSCAEGDTGYVYAELLEFSVKSSSVETMPDLPLKVMMNVGNPDRAFDFACLPNEGVGLARLEFIINRMIGVHPRALLEFDDQEPQLQNEIREMMKGFDSPREFYVGRLTEGIATLGAAFYPKRVIVRLSDFKSNEYANLVGGERYEPDEENPMLGFRGAGRYVSDSFRDCFALECEAVKRVRNDMGLTNVEIMIPFVRTVDQAKAVVEELARQGLKRGENGLKIIMMCEIPSNALLAEQFLEYFDGFSIGSNDMTQLALGLDRDSGVVSELFDERNDAVKALLSMAIRAAKKQGKYVGICGQGPSDHEDFAAWLMEEGIDSLSLNPDTVVQTWLSLAELKK

SEQ ID NO: 7 - DNA, 50 bp, Artificial Sequence
  Primer
atgtccaacaatggctcgtcaccgctggtggtgtaggctggagctgcttc

SEQ ID NO: 8 - DNA, 50 bp, Artificial Sequence
  Primer
ttatttcttcagttcagccaggcttaaccactgtcaaacatgagaattaa

SEQ ID NO: 9 - DNA, 22 bp, Artificial Sequence
  Primer
attgtcgaagagagcagatttg

SEQ ID NO: 10 - DNA, 22 bp, Artificial Sequence
  Primer
ttaacgcaggatgtctgtgaag

SEQ ID NO: 11 - DNA, 69 bp, Artificial Sequence
  Primer
atgtccaataaaggcgaacagccgttagtactttggtacaaccagcttggtgtaggctggagctgcttc

SEQ ID NO: 12 - DNA, 69 bp, Artificial Sequence
  Primer
tcaggcgggtttgtttagcgccgccaggcttaaccaggtttccaccacgctgtcaaacatgagaattaa

SEQ ID NO: 13 - DNA, 22 bp, Artificial Sequence
  Primer
ggcgcaattctgaaactgtgtc

SEQ ID NO: 14 - DNA, 22 bp, Artificial Sequence
  Primer
aaagaattgctgtgtgtagtct

SEQ ID NO: 15 - DNA, 50 bp, Artificial Sequence
  Primer
atgtccaacaatggctcgtcaccgctggtgcctcacgctgccgcaagcac

SEQ ID NO: 16 - DNA, 51 bp, Artificial Sequence
  Primer
ttatttcttcagttcagccaggcttaaccaagaacaactgttcaccgttag

SEQ ID NO: 17 - DNA, 20 bp, Artificial Sequence
  Primer
caaaccgttcatttatcaca

SEQ ID NO: 18 - DNA, 21 bp, Artificial Sequence
  Primer
catcttcggggatcacataac

(19) United States
(12) Patent Application Publication
(10) Pub. No.: US 2023/0265473 A1
(43) Pub. Date: Aug. 24, 2023

(54) IMPROVED CYSTEINE-PRODUCING STRAINS

(71) Applicant: WACKER CHEMIE AG, Munich (DE)

(72) Inventors: Rupert PFALLER, Munich (DE); Johanna KOCH, Munich (DE)

(73) Assignee: WACKER CHEMIE AG, Munich (DE)

(21) Appl. No.: 18/012,296

(22) PCT Filed: Jun. 26, 2020
(86) PCT No.: PCT/EP2020/068021

§ 371 Date: Dec. 22, 2022

(51) Int. Cl. C12P 13/12; C12N 1/20; C12N 9/12; C12N 9/04; C12N 9/10; C12N 15/70

(52) U.S. Cl. CPC C12P 13/12; C12N 1/20; C12N 9/1294; C12N 9/0006; C12N 9/1029; C12N 15/70; C12Y 207/09002; C12R 2001/19

(57) ABSTRACT

Genetically modified microorganism strains for the fermentative production of cysteine provide higher yields of L-cysteine or L-cystine during fermentation. Cysteine production is improved in the genetically modified microorganism strains by attenuating or inactivating phosphoenolpyruvate synthase enzyme activity, alone or in combination with the overexpression of efflux proteins and proteins that reduce feedback inhibition by cysteine and by serine.

### CROSS REFERENCE TO RELATED APPLICATIONS

This application is the U.S. National Phase of PCT/EP2020/068021 filed Jun. 26, 2020, the disclosure of which is incorporated in its entirety by reference herein.

### SEQUENCE LISTING

The text file ppsa_mutante_sequence_listing_st25 of size 33 KB created Dec. 2, 2022 filed herewith, is hereby incorporated by reference.

### BACKGROUND

### 1. Field of the Invention

The present disclosure relates to a microorganism strain suitable for fermentative production of L-cysteine, wherein the relative enzyme activity in the strain of the enzyme class identified by the number EC 2.7.9.2 in the KEGG database is inactivated or is reduced in relation to the specific activity of the wild-type enzyme, and wherein the strain forms an increased amount of L-cysteine compared to the microorganism strain having wild-type enzyme activity of the enzyme class identified by the number EC 2.7.9.2 in the KEGG database, and wherein the gene encoding said enzyme activity is identified by ppsA. Furthermore, the present disclosure provides a process for producing L-cysteine using the cells from the microorganism strain.

### 2. DESCRIPTION OF THE RELATED ART

Cysteine, abbreviated Cys or C, is an α-amino acid having the side chain —CH₂—SH. Since the naturally occurring enantiomeric form is L-cysteine and since only the L enantiomer is a proteinogenic amino acid, unless the D enantiomer is specifically mentioned, L-cysteine is the enantiomer that is meant in the present disclosure when the term cysteine is used. Oxidation of the sulfhydryl groups can result in two cysteine residues forming a disulfide bond with each other to form cystine. Unless the D enantiomer is specifically mentioned, the L-enantiomer (or L-cystine, or (R,R)-3,3′-dithiobis(2-aminopropionic acid)) is the enantiomer that is meant in the present disclosure. L-cysteine is a semi-essential amino acid for humans, since it can be formed from the amino acid methionine.

In all organisms, cysteine occupies a key position in sulfur metabolism and is used in the synthesis of proteins, glutathione, biotin, lipoic acid, thiamine, taurine, methionine and other sulfur-containing metabolites. Moreover, L-cysteine serves as a precursor for the biosynthesis of coenzyme A.

The biosynthesis of cysteine has been studied in detail in bacteria, especially in enterobacteria. A summary of cysteine biosynthesis can be found in Wada and Takagi, Appl. Microbiol. Biotechnol. (2006) 73: 48-54.

The amino acid L-cysteine is of economic importance.

It is used for example as a food additive (particularly in the baking industry), as a raw material in cosmetics, and as a starting material for the production of active pharmaceutical ingredients (particularly N-acetylcysteine and S-carboxymethylcysteine).

Besides classic preparation of cysteine by means of extraction from keratin-containing material such as hair, bristles, horns, hoofs and feathers, or by means of biotransformation through enzymatic conversion of precursors, there is also a process for fermentative production of cysteine. The prior art with regard to the fermentative preparation of cysteine using microorganisms is, for example, disclosed in EP 0 858 510 B1, EP 0 885 962 B1, EP 1 382 684 B1, EP 1 220 940 B2, EP 1 769 080 B1 and EP 2 138 585 B1. The bacterial host organisms that are used include strains of the genus Corynebacterium and members of the Enterobacteriaceae family, such as, for example, Escherichia coli or Pantoea ananatis.

Various methods are available to improve cysteine production in a microorganism strain. In addition to the classic approach of obtaining improved cysteine producers by mutation and selection, the strains may also be specifically genetically modified to achieve effective overproduction of cysteine.

For instance, the introduction of a cysE allele which encodes a serine O-acetyltransferase having reduced feedback inhibition by cysteine led to an increase in cysteine production (EP 0 858 510 B1; Nakamori et al., Appl. Env. Microbiol. (1998) 64: 1607-1611). A feedback-resistant CysE enzyme largely decouples the formation of O-acetyl-L-serine, the direct precursor of cysteine, from the cysteine level in the cell.

O-Acetyl-L-serine is formed from L-serine and acetyl-CoA. Therefore, it is of great importance to provide L-serine in sufficient quantity for cysteine production. This can be achieved by introducing a serA allele encoding a 3-phosphoglycerate dehydrogenase having reduced feedback inhibitability by L-serine. As a result, the formation of 3-hydroxypyruvate, a biosynthetic precursor of L-serine, is largely decoupled from the L-serine level in the cell. Examples of such SerA enzymes are described in EP 0 620 853 B1 and EP 1 496 111 B 1. Alternatively, Bell et al., Eur. J. Biochem. (2002) 269: 4176-4184 disclose modifications to the serA gene to deregulate enzyme activity.

Furthermore, it is known that the cysteine yield in fermentation can be increased by attenuating or destroying genes encoding cysteine-degrading enzymes, such as, for example, the tryptophanase TnaA or the cystathionine β-lyases MalY or MetC (EP 1 571 223 B1).

Increasing the transport of cysteine out of the cell is another way to increase the product yield in the medium. This can be achieved by overexpressing so-called efflux genes. These genes encode membrane-bound proteins which mediate the export of cysteine out of the cell.

Various efflux genes for cysteine export have been described (EP 0 885 962 B1, EP 1 382 684 B1). The export of cysteine out of the cell into the fermentation medium has several advantages:
1) L-cysteine is continuously withdrawn from the intracellular reaction equilibrium, thereby keeping the level of this amino acid in the cell low and causing the feedback inhibition of sensitive enzymes by L-cysteine to cease:
   (1) L-cysteine (intracellular)<->L-cysteine (medium)
2) The L-cysteine secreted into the medium is oxidized to form the disulfide L-cystine in the presence of oxygen, which is introduced into the medium during cultivation (EP 0 885 962 B1):
   (2) 2 L-cysteine+½O₂->L-cystine+H₂O
   Since the solubility of L-cystine in aqueous solution at a neutral pH is very low compared to cysteine, the disulfide already precipitates at a low concentration and forms a white precipitate:
   (3) L-cystine (dissolved)->L-cystine (precipitate)
   The precipitation of L-cystine lowers the level of the product dissolved in the medium, thereby also causing the reaction equilibrium of (1) and that of (2) to be pulled to the product side.
3) Purifying the product is significantly less complex if the amino acid can be obtained directly from the fermentation medium than if the product accumulates intracellularly and cell disruption is required in order to access the amino acid.

Besides the genetic modification of a cysteine producing strain, the optimization of the fermentation process, i.e. how the cells are cultivated, also plays an important role in the development of an efficient production process. Various cultivation parameters, such as, for example, the nature and metering of the carbon and energy source, the temperature, the supply of oxygen (EP 2 707 492 B1), the pH and the composition of the culture medium, can influence the product yield and/or the product spectrum in the fermentative production of cysteine.

Since raw material and energy costs are constantly rising, there is a constant need to increase the product yield in cysteine production so as to improve the economic viability of the process.

### SUMMARY OF THE INVENTION

It is an object of the present invention to provide a microorganism strain for fermentative production of cysteine, from which higher yields of L-cysteine or L-cystine can be produced during fermentation compared to known strains from the prior art.

The object is achieved by a microorganism strain suitable for fermentative production of L-cysteine, wherein the relative enzyme activity of the enzyme class identified by the number EC 2.7.9.2 in the KEGG database is inactivated or is reduced in relation to the specific activity of the wild-type enzyme, and wherein the microorganism strain forms an increased amount of L-cysteine compared to the microorganism strain having wild-type enzyme activity of the enzyme class identified by the number EC 2.7.9.2 in the KEGG database, and wherein the gene encoding said enzyme activity is identified by ppsA.

The enzyme activity of the enzyme class identified by the number EC 2.7.9.2 in the KEGG database is defined by the ability of its members to produce pyruvate from phosphoenolpyruvate in a reversible reaction according to the formula:
(4) phosphoenolpyruvate+phosphate+AMP<->pyruvate+H₂O+ATP
(AMP: adenosine monophosphate; ATP: adenosine triphosphate)

This enzyme activity is therefore also referred to as phosphoenolpyruvate synthase (PEP synthase, EC 2.7.9.2) or else synonymously as pyruvate-H₂O dikinase. The gene encoding this protein is abbreviated as ppsA in the context of this invention.

### DETAILED DESCRIPTION OF THE INVENTION

Detection of enzyme activity (enzyme assay, PEP synthase assay):

The PEP synthase activity of a microorganism strain may be determined by pelleting the cells from the culture in a liquid medium, washing the cells, and preparing a cell extract, for example with the aid of a FastPrep-24™ 5G cell homogenizer (MP Biomedicals). The protein content of the extract may, for example, be determined by means of the “Qubit® Protein Assay Kit” (Thermo Fisher Scientific).

PEP synthase enzyme activity may be measured by the stoichiometric production of phosphate from the reaction of pyruvate and ATP according to equation (4), for example with the aid of the “Malachite Green Phosphate Assay Kit” (Sigma Aldrich). Alternatively, the stoichiometric production of AMP or phosphoenolpyruvate or the stoichiometric consumption of pyruvate or ATP may also be determined (cf. equation 4). An assay for determining PEP synthase enzyme activity via the ATP-dependent consumption of pyruvate is, for example, described in Berman and Cohn, J. Biol. Chem. (1970) 245: 5309-5318. Likewise described in Berman and Cohn, J. Biol. Chem. (1970) 245: 5309-5318 is an assay for the ATP-dependent formation of phosphoenolpyruvate.

Specific enzyme activity is calculated by basing the enzyme activity on 1 mg of total protein of the cell extract without any further purification or treatment (U/mg protein). Comparison of different PEP synthase enzymes requires that the cell extract be prepared in the same way. As already described, the cell extract may, for example, be prepared with the aid of a FastPrep24™ 5G cell homogenizer (MP Biomedicals).

Alternatively, different enzymes may be compared by also basing the specific activity on 1 mg of the enzymes respectively purified in the same way (U/mg purified protein). A method for purifying PEP synthase and for determining the specific activity of the purified protein is, for example, described in Berman and Cohn, J. Biol. Chem. (1970) 245: 5309-5318.

Relative enzyme activity may be determined by setting to 100% the specific enzyme activity determined in the PEP synthase assay of the microorganism strain bearing the Wt allele in relation to the gene encoding the PEP synthase. The value measured in the PEP synthase assay for the specific enzyme activity of a sample is specified as a percentage in relation to this strain with Wt enzyme.

The term “Open reading frame” (ORF), which is synonymous with cds or coding sequence refers to the region of DNA or RNA that begins with a start codon and ends with a stop codon and encodes the amino acid sequence of a protein. The ORF is also referred to as the coding region or structural gene.

The term “Gene” refers to the section of DNA that contains all the basic information for producing a biologically active RNA. A gene contains the section of DNA from which a single-stranded RNA copy is produced by transcription and also the expression signals involved in the regulation of this copying process. The expression signals include for example at least one promoter, a transcription start site, a translation start site, and a ribosome binding site. In addition, a terminator and one or more operators are possible as expression signals.

In the context of this disclosure, proteins, for example PpsA, start with a capital letter, whereas the DNA sequences encoding said proteins (cds) are identified by a lowercase letter (e.g., ppsA).

Accordingly, E. coli ppsA refers to the cds of the ppsA gene from E. coli as specified in SEQ ID NO: 1 from nucleotide 333-2711. E. coli PpsA refers to the protein encoded by said cds (E. coli ppsA) and specified in SEQ ID NO: 2. The protein is a phosphoenolpyruvate synthase.

P. ananatis ppsA refers to the cds of the ppsA gene from P. ananatis as specified in SEQ ID NO: 3 from nucleotide 417-2801. P. ananatis PpsA refers to the protein encoded by said cds (P. ananatis ppsA) and specified in SEQ ID NO: 4.

The abbreviation WT (Wt) refers to the wild type. The term “Wild-type gene” refers to the form of the gene that arose naturally through evolution and is present in the wild-type genome of an organism. The DNA sequence of Wt genes is publicly accessible in databases such as NCBI.

Alleles define the states of a gene that can be converted into one another by mutation, i.e., by changes to the nucleotide sequence of the DNA. The gene that occurs naturally in a microorganism is referred to as the wild-type allele and the variants derived therefrom are referred to as the mutated allele of the gene.

Homologous genes or homologous sequences are understood to mean that the DNA sequences of said genes or sections of DNA may have at least about 80% sequence identity to each other as determined using the methods described below. For example, the DNA sequences of homologous genes or sections of DNA may have at least about 70%, or at least about 75%, or at least about 80%, or at least about 85%, or at least about 90%, or at least about 95%, or more sequence identity to each other.

The degree of DNA identity is determined by the “nucleotide blast” program which can be found at http://blast.ncbi.nih.gov/ and which is based on the blastn algorithm. The default parameters were used as the algorithm parameters for an alignment of two or more nucleotide sequences. The default general parameters are: Max target sequences=100; Short queries=“Automatically adjust parameters for short input sequences”; Expect Threshold=10; Word size=28; Automatically adjust parameters for short input sequences=0. The corresponding default scoring parameters are: Match/Mismatch Scores=1,−2; Gap Costs=Linear.

Protein sequences are compared using the “protein blast” program at http://blast.ncbi.nlm.nih.gov/. This program uses the blastp algorithm. The default parameters were used as the algorithm parameters for an alignment of two or more protein sequences. The default general parameters are: Max target sequences=100; Short queries=“Automatically adjust parameters for short input sequences”; Expect Threshold=10; Word size=3; Automatically adjust parameters for short input sequences=0. The default scoring parameters are: Matrix=BLOSUM62; Gap Costs=Existence: 11 Extension: 1; Compositional adjustments=Conditional compositional score matrix adjustment.

In the microorganisms according to the present disclosure, the relative enzyme activity of the enzyme class identified by the number EC 2.7.9.2 in the KEGG database may be inactivated or reduced in relation to the specific activity of the wild-type enzyme by at least about 10%. In at least some preferred embodiments, the relative enzyme activity of the enzyme class identified by the number EC 2.7.9.2 in the KEGG database may be inactivated or reduced in relation to the specific activity of the wild-type enzyme by at least about 25%, or by at least about 60%, or by at least about 70% in order of preference. For example, the relative enzyme activity of the enzyme class identified by the number EC 2.7.9.2 in the KEGG database may be inactivated or reduced in relation to the specific activity of the wild-type enzyme by at least about 10%, or at least about 15%, or at least about 20%, or at least about 25%, or at least about 30%, or at least about 35%, or at least about 40%, or at least about 45%, or at least about 50%, or at least about 60%, or at least about 65%, or at least about 70%, or at least about 75%, or at least about 80%, or at least about 85%, or at least about 90%, or at least about 95%, or more. An enzyme activity of the enzyme encoded by the ppsA gene that is reduced by at least 10% (or 25%/60%/70%) is also referred to as a residual activity of at most 90% (or 75%/40%/30%).

In a preferred embodiment, the microorganism strain is characterized in that it no longer has any enzyme activity of the enzyme class identified by the number EC 2.7.9.2 in the KEGG database, i.e., the relative enzyme activity of the enzyme class identified by the number EC 2.7.9.2 in the KEGG database is reduced by 100% in relation to the specific activity of the wild-type enzyme.

In the context of the present disclosure, “compared to the/in comparison with the/in relation to the (corresponding) wild-type enzyme” means in comparison with the activity of the protein encoded by the nonmutated form of the gene from a microorganism, i.e., by the gene which arose naturally through evolution and is present in the wild-type genome of said microorganism.

The microorganism strains suitable for fermentative production of L-cysteine include all microorganisms which contain a deregulated biosynthetic metabolic pathway (homologous or heterologous) which leads to the synthesis of cysteine, cystine or derivatives derived therefrom. Such strains are, for example, disclosed in EP 0 885 962 B1, EP 1 382 684 B1, EP 1 220 940 B2, EP 1 769 080 B1 and EP 2 138 585 B1.

The microorganisms suitable for fermentative production of L-cysteine are preferably characterized in that they have one of the following modifications:
a) The microorganism strains are distinguished by a modified 3-phosphoglycerate dehydrogenase (serA) having a feedback inhibition by L-serine that is reduced by a factor of at least two in comparison with the corresponding wild-type enzyme (as described, for example, in EP 1 950 287 B1).
   In comparison with the corresponding wild-type enzyme, particularly preferred variants of the 3-phosphoglycerate dehydrogenase (serA) have a feedback inhibition by L-serine that is reduced by a factor of from at least about 5 to at least about 50. For example, particularly preferred variants of the 3-phosphoglycerate dehydrogenase (serA) have a feedback inhibition by L-serine that is reduced in comparison with the corresponding wild-type enzyme by a factor of at least about 5, or at least about 10, or at least about 15, or at least about 20, or at least about 25, or at least about 30, or at least about 35, or at least about 40, or at least about 45, or at least about 50. Preferably, particularly preferred variants of the 3-phosphoglycerate dehydrogenase (serA) have a feedback inhibition by L-serine that is reduced in comparison with the corresponding wild-type enzyme by a factor of at least about 10. More preferably, particularly preferred variants of the 3-phosphoglycerate dehydrogenase (serA) have a feedback inhibition by L-serine that is reduced in comparison with the corresponding wild-type enzyme by a factor of at least about 50.
b) The microorganism strains contain a serine 0-acetyltransferase (cysE) which, in comparison with the corresponding wild-type enzyme, has a feedback inhibition by cysteine that is reduced by a factor of at least two (as described, for example, in EP 0 858 510 B1 or Nakamori et al., Appl. Env. Microbiol. (1998) 64: 1607-1611).
   In comparison with the corresponding wild-type enzyme, particularly preferred variants of the serine 0-acetyltransferase (cysE) have a feedback inhibition by cysteine that is reduced by a factor of from at least about 5 to at least 50, or preferably from at least about 10 to at least 50, or more preferably by at least about 50. For example, particularly preferred variants of the serine 0-acetyltransferase (cysE) have a feedback inhibition by cysteine that is reduced in comparison with the corresponding wild-type enzyme by a factor of at least about 5, or at least about 10, or at least about 15, or at least about 20, or at least about 25, or at least about 30, or at least about 35, or at least about 40, or at least about 45, or at least about 50.
c) The microorganism strains exhibit, in comparison with the corresponding wild-type cell, an export of cysteine out of the cell that is increased by a factor of at least two due to overexpression of an efflux gene.
   In comparison with a wild-type cell, the overexpression of an efflux gene preferably leads to an export of cysteine out of the cell that is increased by a factor of from at least about 5 to at least about 20, or preferably from at least about 10 to at least about 20, or more preferably by at least 20. For example, the overexpression of an efflux gene preferably leads to an export of cysteine out of the cell that is increased by a factor of at least about 5, or at least about 10, or at least about 15, or at least about 20.
   The efflux gene preferably comes from the group consisting of ydeD (see EP 0 885 962 B 1), yfiK (see EP 1 382 684 B 1), cydDC (see WO 2004/113373 A1), bcr (see US 2005-221453 AA) and emrAB (see US 2005-221453 AA) from E. coli or the correspondingly homologous gene from a different microorganism.

Such strains are, for example, known from EP 0 858 510 B1 and EP 0 885 962 B1.

Furthermore, the microorganism strains suitable for fermentative production of L-cysteine preferably comprise at least one cysteine-degrading enzyme that is attenuated to such an extent that the cell only contains at most about 50% of this enzyme activity in comparison to a wildtype cell. For example, the cell may contain about 0%, or about 5%, or about 10%, or about 15%, or about 20%, or about 25%, or about 30%, or about 35%, or about 40%, or about 45%, or about 50% of cysteine-degading enzyme activity as compared with a wild-type cell. The cysteine-degrading enzyme is preferably selected from the group consisting of tryptophanase (TnaA) and cystathionine β-lyase (MalY, MetC).

The microorganism strains suitable for fermentative production of L-cysteine that are described in the previous paragraphs are deregulated with respect to their cysteine metabolism in such a way that they produce an increased amount of L-cysteine as compared to the microorganism strain which is not deregulated with respect to cysteine metabolism and which has wild-type enzyme activity of the enzyme class identified by the number EC 2.7.9.2 in the KEGG database. Since the cells of a microorganism strain, which is not deregulated with respect to cysteine metabolism and which has wild-type enzyme activity of the enzyme class identified by the number EC 2.7.9.2 in the KEGG database, have approximately 0 g/l L-cysteine in the culture (cf. Table 2), an increased amount of L-cysteine in the culture includes any amount exceeding 0.05 g/l L-cysteine measured in the culture after 24 hours of cultivation.

Preferably, the microorganism strain is a strain from the Enterobacteriaceae or Corynebacteriaceae family, preferably a strain from the Enterobacteriaceae family. Such strains are commercially available from, inter alia, the DSMZ-German Collection of Microorganisms and Cell Cultures GmbH (Braunschweig) for example.

Preferably, the microorganism strain is selected from the group consisting of Escherichia coli, Pantoea ananatis and Corynebacterium glutamicum. In more preferred embodiments the microorganism strain is selected from the group consisting of Escherichia coli and Pantoea ananatis. In the most preferred embodiments, the microorganism strain is a strain of the species Escherichia coli.

The E. coli strain is preferably selected from E. coli K12, and more specifically is E. coli K12 W3110. Such strains are commercially available from, inter alia, the DSMZ-German Collection of Microorganisms and Cell Cultures GmbH (Braunschweig) for example, including E. coli K12 W3110 DSM 5911 (id. ATCC 27325) and Pantoea ananatis DSM 30070 (id. ATCC 11530). PpsA is preferably PpsA from E. coli having the protein sequence of SEQ ID NO: 2 or PpsA from P. ananatis having the protein sequence of SEQ ID NO: 4.

In a preferred embodiment, the microorganism strain comprises at least one mutation in the ppsA gene and produces an increased amount of L-cysteine in comparison with wild-type cells. Preferably, the genetic modification in the ppsA gene causes the protein expressed by said gene to have a reduced relative enzyme activity of the enzyme class identified by the number EC 2.7.9.2 in the KEGG database, in relation to the specific activity of the wild-type enzyme, or to have no such enzyme activity.

Furthermore, the production strain according to the present disclosure may be further optimized for further improvement of cysteine production.

Optimization may, for example, be achieved genetically by additionally expressing one or more genes suitable for improving production properties. Said genes may be expressed in the production strain in a manner known per se either as separate gene constructs or in combination as an expression unit (as a so-called operon).

Furthermore, the production strain may be optimized by inactivating yet further genes, including gene products that have an adverse effect on cysteine production, in addition to the ppsA gene.

However, optimization is also possible through random mutagenesis and selection of strains having improved cysteine production.

In the context of the present disclosure, genetic modifications in the ppsA gene are defined as follows:
a) the coding sequence of the ppsA gene is partially or completely deleted,
b) the coding sequence of the ppsA gene is modified by one or more insertions or 5′ and/or 3′ elongations,
c) the ppsA structural gene contains one or more mutations, especially point mutations, that result in the expressed phosphoenolpyruvate synthase having attenuated enzyme activity or being completely inactive,
d) the ppsA structural gene contains one or more mutations, especially point mutations, that result in strong attenuation or complete suppression of ppsA expression or a reduction in mRNA stability, or
e) the expression of the ppsA gene or the translation of the ppsA mRNA is attenuated or completely suppressed due to genetic modifications to the 5′ and/or 3′ noncoding ppsA sequences (promoter, 5′-UTR, Shine-Dalgarno sequence and/or terminator)
   and the protein expressed by this sequence has a reduced relative PEP synthase enzyme activity in relation to the specific activity of the wild-type enzyme.

In the context of the present disclosure, any combination of the genetic modifications in the ppsA gene that are listed in a) to e) is also possible. In summary, it is thus possible in the context of the present disclosure for less PpsA protein, or none, to be formed and/or for the expressed PpsA protein to be less active or inactive.

In an alternative approach, it is also possible to attenuate or completely inactivate ppsA enzyme activity at the level of gene transcription by a so-called “anti-sense RNA” strategy known to a person skilled in the art. It is also conceivable for ppsA enzyme activity to be attenuated or completely inactivated by adding an inhibitor, including a chemical inhibitor or protein inhibitor.

Preferably, modifying the ppsA gene in the strain according to the invention involves complete or partial deletion of the ppsA structural gene, mutation of the ppsA structural gene that causes attenuation of enzyme activity or enzyme inactivation, or mutation of the ppsA structural gene and/or untranscribed or untranslated gene regions thereof, which gene regions regulate expression and are on the 5′ and 3′ flanks, wherein such mutations cause attenuation or complete suppression of the expression or translation of the ppsA gene or else a reduction in the stability of ppsA mRNA.

In preferred embodiments, inactivation of the ppsA gene in the strain according to the present disclosure is caused either by complete or partial deletion of the ppsA cds (i.e., in the case of the ppsA cds of E. coli, nucleotides 333-2711 of the DNA sequence of SEQ ID NO: 1, or in the case of the ppsA cds of P. ananatis, nucleotides 417-2801 of the DNA sequence of SEQ ID NO: 3) or by mutation of the ppsA structural gene in a manner causing attenuation of enzyme activity or enzyme inactivation or a reduction in mRNA stability.

In a preferred embodiment, the microorganism strain is characterized in that the mutated gene is selected from the group consisting of the ppsA gene from Escherichia coli, the ppsA gene from Pantoea ananatis, and a gene homologous to these genes. The ppsA gene from E. coli is disclosed in the entry in the NCBI gene database with the gene ID 946209, and the ppsA gene from P. ananatis is disclosed in the entry in the NCBI gene database with the gene ID 11796889. For the term “homologous gene”, the definition given above applies. Preferably, the mutated ppsA gene is the ppsA gene from Escherichia coli. In an additionally preferred embodiment, the cds is the cds of the ppsA gene from E. coli that is disclosed in SEQ ID NO: 1 nucleotides 333-2711 (encoding a protein having SEQ ID NO: 2) or the cds of the ppsA gene from Pantoea ananatis that is disclosed in SEQ ID NO: 3 nucleotides 417-2801 (encoding a protein having SEQ ID NO: 4).

In a preferred embodiment, the microorganism strain is characterized in that the coding DNA sequence of the ppsA gene is SEQ ID NO: 5 or a sequence homologous thereto. For the term “homologous sequence”, the definition given above applies.

In this case, the mutations in the DNA sequence specified in SEQ ID NO: 1 lead to the mutation of the three amino acids in the WT protein sequence specified in SEQ ID NO: 2, namely valine at position 126 mutated to methionine (V126M), arginine at position 427 mutated to histidine (R427H) and valine at position 434 mutated to isoleucine (V434I), in accordance with a ppsA-MHI gene having the DNA sequence as in SEQ ID NO: 5, encoding a ppsA-MHI protein having the amino acid sequence as disclosed in SEQ ID NO: 6.

Various methods for inactivating and mutating the ppsA gene are known to a person skilled in the art. In the simplest case, the parent strain can be subjected to mutagenesis in a known manner (e.g., chemically by means of mutagenic chemicals such as N-methyl-N′-nitro-N-nitrosoguanidine or physically by means of UV irradiation), with mutations being randomly generated in the genomic DNA and the desired ppsA mutant then being selected from the multiplicity of mutants generated, for example after the mutants have each been singularized, by means of the absence of a color reaction based on enzyme activity or by genetic means through detection of a defective ppsA gene.

In contrast to complex random mutagenesis and selection of the sought-after ppsA mutant, the ppsA gene can be subjected to targeted inactivation in a relatively simple manner, for example by the known mechanism of homologous recombination. Cloning systems for targeted gene inactivation by means of homologous recombination are known to a person skilled in the art and commercially available, as disclosed, for example, in the user manual of the “Quick and Easy E. coli Gene Deletion Kit”, based on Red®/ET® technology from Gene Bridges GmbH (see “Technical Protocol, Quick & Easy E. coli Gene Deletion Kit, by Red®)/ET® Recombination, Cat. No. K006, Version 2.3, June 2012” and the references cited therein).

According to the prior art, the ppsA gene or part of the gene can be isolated and foreign DNA can be cloned into the ppsA gene, thereby interrupting the protein-defining open reading frame of the ppsA gene. A DNA construct suitable for the targeted inactivation of the ppsA gene can thus consist of a 5′ section of DNA which is homologous to the genomic ppsA gene, followed by a gene segment comprising the foreign DNA, followed by a 3′ section of DNA which is again homologous to the genomic ppsA gene.

The possible region in the ppsA gene for homologous recombination can thus comprise not just the region encoding phosphoenolpyruvate synthase. The possible region can also comprise DNA sequences which flank the ppsA gene, namely in the 5′ region before the start of the coding region (gene transcription promoter, for example nucleotides 1-332 in SEQ ID NO: 1, or nucleotides 1-416 in SEQ ID NO: 3) and in the 3′ region after the end of the coding region (gene transcription terminator, for example nucleotides 2712-3000 in SEQ ID NO: 1, or nucleotides 2802-3062 in SEQ ID NO: 3), the modification of which by homologous recombination can lead to inactivation of the ppsA gene just like the modification of the coding region.

The foreign DNA is preferably a selection-marker expression cassette. It consists of a gene transcription promoter functionally linked to the actual selection marker gene, optionally followed by a gene transcription terminator. In this case, the selection marker also contains 5′ and 3′ flanking homologous sequences of the ppsA gene.

Preferably, the selection marker has 5′ and 3′ flanking homologous sequences of the ppsA gene that each have a length of from at least about 30 to at least about 50 nucleotides. For example, 5′ and 3′ flanking homologous sequences of the ppsA gene of the selection marker each have a length of at least about 30, or at least about 35, or at least about 40, or at least about 45, or at least about 50 nucleotides. More preferably, 5′ and 3′ flanking homologous sequences of the ppsA gene of the selection marker each have a length of at least about 50 nucleotides.

The DNA construct for inactivating the ppsA gene can thus, starting from the 5′ end, consist of a sequence homologous to the ppsA gene, followed by the expression cassette of the selection marker, for example selected from the class of the antibiotic resistance genes, and followed by a further sequence homologous to the ppsA gene.

In a preferred embodiment, the DNA construct for inactivating the ppsA gene consists, starting from the 5′ end, of a sequence homologous to the ppsA gene of from at least about 30 nucleotides to at least about 50 nucleotides in length, followed by the expression cassette of the selection marker, selected from the class of the antibiotic resistance genes, and followed by a further sequence homologous to the ppsA gene of at least about 30 nucleotides to at least about 50 nucleotides in length.

The selection marker genes are generally genes, the gene product of which enables the parent strain to grow under selective conditions under which the original parent strain cannot grow.

Preferred selection marker genes are selected from the group of the antibiotic resistance genes such as, for example, the ampicillin resistance gene, the tetracycline resistance gene, the kanamycin resistance gene, the chloramphenicol resistance gene or the neomycin resistance gene. Other preferred selection marker genes allow parent strains having a metabolic defect (e.g., amino acid auxotrophies) to grow under selective conditions as a result of correction of the metabolic defect by expression of said selection marker genes. Lastly, another possibility is selection marker genes, the gene product of which chemically alters an inherently toxic compound for the parent strain and thus inactivates said compound (e.g., the gene of the enzyme acetamidase, which splits the compound acetamide, toxic for many microorganisms, into the nontoxic products acetate and ammonia).

The ampicillin resistance gene, the tetracycline resistance gene, the kanamycin resistance gene and the chloramphenicol resistance gene, particularly the tetracycline resistance gene and the kanamycin resistance gene are preferred among the selection marker genes.

There are also systems based on homologous recombination that, in addition to targeted gene inactivation, also provide the option of removing the selection marker from the genome, thereby making it possible to produce double and multiple mutants. Such a system is, for example, so-called Lambda Red technology, commercially available as the “Quick and Easy E. coli Gene Deletion Kit”, based on Red®/ET® technology from Gene Bridges GmbH (see “Technical Protocol, Quick & Easy E. coli Gene Deletion Kit, by Red®/ET® Recombination, Cat. No K006, Version 2.3, June 2012” and the references cited therein).

Examples of strains according to the invention having an inactivated ppsA gene are the strains E. coli W3110-ΔppsA and P. ananatis-ΔppsA::kan that are disclosed in the examples. Both strains are characterized in that their ppsA gene has been inactivated by homologous recombination.

Another such system for targeted gene inactivation based on homologous recombination is a method for gene inactivation or genetic modification that is known to a person skilled in the art and described in Example 3 and that is based on a combination of Lambda Red recombination with counter-selection screening. Said system is, for example, described in Sun et al., Appl. Env. Microbiol. (2008) 74: 4241-4245. A DNA construct is used to inactivate, for example, the ppsA gene, consisting (starting from the 5′ end) of a sequence homologous to the ppsA gene, followed by two expression cassettes in any order, consisting of a) an expression cassette of the selection marker selected from the class of the antibiotic resistance genes and b) an expression cassette of the sacB gene encoding the enzyme levansucrase, and lastly followed by a further sequence homologous to the ppsA gene.

In a first step, the DNA construct is transformed into the production strain and antibiotic-resistant clones are isolated. The clones obtained are distinguished by the fact that they cannot grow on sucrose due to the coincorporated sacB gene. The two marker genes can be removed by the principle of counter-selection, in that, in a second step, a suitable DNA fragment replaces the two marker genes by homologous recombination. The clones obtained in this step then regain their ability to grow on sucrose and then also regain their sensitivity to the antibiotic. This method is used in Example 3 to exchange the ppsA WT gene of E. coli (SEQ ID NO: 1) for the triple mutant ppsA-MHI (SEQ ID NO: 5) that is described below.

The E. coli strain W3110-ppsA-MHI is disclosed in the examples as an example of a strain according to the invention that has attenuated PpsA enzyme activity due to a mutation of the coding sequence of the ppsA gene. W3110-ppsA-MHI contains the cds of the PpsA triple mutant PpsA-V126M-R427H-V434I (ppsA-MHI). The cds of the mutated gene of ppsA-MHI corresponds to the DNA sequence SEQ ID NO: 5 and encodes a PpsA protein having sequence SEQ ID NO: 6. The PpsA-MHI protein comprises a protein having sequence SEQ ID NO: 6 with the following changes in the amino acid sequence (changed from the WT sequence specified in SEQ ID NO: 2): valine at position 126 is mutated to methionine (V126M), arginine at position 427 is mutated to histidine (R427H), and valine at position 434 is mutated to isoleucine (V434I).

Due to these mutations, the PpsA-MHI protein exhibited a relative enzyme activity of 26.8% in comparison with the specific wild-type enzyme activity (see Example 5, Table 1).

In case of the E. coli ppsA gene, it is preferred that at least one of the mutations in the cds leads to at least one of the following changes in the amino acid sequence in SEQ ID NO: 2: valine at position 126, arginine at position 427 and/or valine at position 434, wherein any of the three amino acids can be exchanged for any other amino acid. Particular preference is given to mutations which lead to the simultaneous mutation of the three amino acids in the amino acid sequence of the WT protein that is specified in SEQ ID NO: 2.

The mutations in the ppsA-MHI gene according to the present disclosure are introduced into the ppsA WT gene using for example, a method known in the art as “site-directed” mutagenesis using a commercially available cloning kit, as disclosed, for example, in the user manual for the “QuickChange II Site-Directed Mutagenesis Kit” from Agilent. Alternatively, the ppsA-MHI gene according to the present disclosure can also be produced in a known manner by DNA synthesis.

The strain according to the present disclosure, comprising a mutated ppsA structural gene wherein the mutation causes attenuation of enzyme activity, for example the E. coli ppsA-MHI triple mutant, can be produced by using the above-described combination of Lambda Red recombination with counter-selection screening for genetic modification (see, for example, Sun et al., Appl. Env. Microbiol. (2008) 74: 4241-4245), as disclosed in the examples. Particularly preferred strains are E. coli W3110 ΔppsA (described in Example 1) and E. coli W3110 ppsA-MHI (described in Example 3).

The present disclosure further provides a fermentative process for producing L-cysteine, characterized in that the microorganism cells according to the present disclosure are used.

The primary product of the process according to the present disclosure that is formed is L-cysteine, from which the compounds L-cystine and thiazolidine can be formed. L-cystine and thiazolidine are formed during fermentation and accumulate both in the culture supernatant and in the precipitate. Thiazolidine is 2-methyl-2,4-thiazolidinedicarboxylic acid, an adduct of cysteine and pyruvate that can be formed as a by-product of cysteine production (EP 0 885 962 B1).

In the context of the present disclosure, the yield of total cysteine is defined as the sum total of the cysteine, cystine and thiazolidine produced. This total is determined from the entire culture, as described in Example 7. It can, for example, be quantified with the aid of the colorimetric assay by Gaitonde (Gaitonde, M. K. (1967) Biochem. J. 104, 627-633).

The prior art does not disclose any processes or production strains in which the production of an amino acid, in particular of cysteine, can be improved by attenuating or inactivating phosphoenolpyruvate synthase enzyme activity.

As shown in the examples of the present application, the attenuation or inactivation of the ppsA enzyme activity in a microorganism strain suitable for cysteine production significantly increases the yields of total cysteine, i.e., the sum total of the cysteine, cystine and thiazolidine produced, in a fermentative process. From the prior art, this was completely unexpected.

As summarized in Table 4 of Example 7, it was surprising that significantly higher cysteine yields were achieved in the fermentation of the ppsA mutants of E. coli W3110 in comparison with the corresponding wild-type strain. Contrary to the prior art and unexpectedly for a person skilled in the art, the attenuation or inactivation of phosphoenolpyruvate synthase activity led to improved cysteine-producing strains.

This novel and inventive measure for improving cysteine-producing strains was confirmed by the results summarized in Table 2 and Table 3 of Example 6, in which the inactivation of the ppsA gene, or a mutated ppsA gene resulting in a PpsA enzyme having attenuated enzyme activity, in Escherichia coli and the inactivation of the ppsA gene in Pantoea ananatis already led to improved cysteine yields in cultivation in shake flasks.

For a person skilled in the art, the attenuation or inactivation of phosphoenolpyruvate synthase activity is therefore a novel useful measure for improving cysteine production in other cysteine-producing strains as well.

Accordingly, in the microorganism strain disclosed herein which is suitable for cysteine production, the enzyme activity of the protein encoded by the ppsA gene in the production strain is attenuated or completely inhibited and, at the same time, cysteine production is increased. Example 7 demonstrates that a strain which is capable of cysteine production and encodes the ppsA mutant ppsA-MHI having reduced PpsA enzyme activity instead of the Wt enzyme achieves significantly higher cysteine yields in fermentation than a strain containing a ppsA WT gene.

In the fermentative process in question, what are formed are not only biomass of the production strain, but also cysteine and its oxidation product cystine. The formation of biomass and cysteine can be correlated temporally. Alternatively, biomass and cysteine can be formed over time in a mutually decoupled manner. Cultivation may be achieved by methods commonly used in the art, such as cultivation in shake flasks (laboratory scale) or else by fermentation (production scale).

With regard to a production-scale process by fermentation, the fermentation volume may be an amount greater than about 1 L, or preferably greater than about 10 L, or more preferably greater than about 10,000 L. For example, the fermentation volume may be greater than about 1 L, or about 2 L, or about 3 L, or about 4 L, or about 5 L, or about 10 L, or about 50 L, or about 100 L, or about 500 L, or about 1000 L, or about 5000 L, or about 10,000 L.

Suitable cultivation media include but are not limited to media commonly used in the art to cultivate microbes. Cultivation media may consist of a carbon source, a nitrogen source, and additives such as vitamins, salts and trace elements, and a sulfur source which optimizes cell growth and cysteine production.

Carbon sources include for example, those that can be used by the production strain for formation of cysteine products. These include all forms of monosaccharides, encompassing C6 sugars (hexoses) such as, for example, glucose, mannose, fructose or galactose, and C5 sugars (pentoses) such as, for example, xylose, arabinose or ribose.

The production process as disclosed herein additionally may include carbon sources in the form of disaccharides, in particular sucrose, lactose, maltose or cellobiose.

Furthermore, the production process according to the present disclosure may also include carbon sources in the form of higher saccharides, glycosides or carbohydrates having more than two sugar units such as, for example, maltodextrin, starch, cellulose, hemicellulose, pectin or monomers or oligomers (enzymatically or chemically) released therefrom by hydrolysis. The hydrolysis of the higher carbon sources may be upstream of the production process according to the invention or alternatively, may take place in situ during the production process.

Additional usable carbon sources other than sugars or carbohydrates may include acetic acid (or acetate salts derived therefrom), ethanol, glycerol, citric acid (and salts thereof) or pyruvate (and salts thereof). However, the use of gaseous carbon sources such as carbon dioxide or carbon monoxide is also conceivable.

Suitable carbon sources that may be used in the production process may also include both pure substances that have been isolated, or to increase economic efficiency, mixtures of the individual carbon sources with no further purification, as can be obtained by chemical or enzymatic digestion of vegetable raw materials such as hydrolysates. These include, for example, hydrolysates of starch (glucose monosaccharide), of sugar beet (glucose, fructose and arabinose monosaccharides), of sugar cane (sucrose disaccharide), of pectin (galacturonic acid monosaccharide) or else of lignocellulose (glucose monosaccharide from cellulose, xylose, arabinose, mannose and galactose monosaccharides from hemicellulose, and noncarbohydrate lignin). Furthermore, waste products from the digestion of vegetable raw materials can also be used as carbon sources, for example molasses (sugar beet) or bagasse (sugar cane).

Preferred carbon sources for cultivating the production strains may include glucose, fructose, sucrose, mannose, xylose, arabinose, and vegetable hydrolysates that can be obtained from starch, lignocellulose, sugar cane or sugar beet.

Glucose and sucrose, either in isolated form or as constituent of a vegetable hydrolysate are particularly preferred carbon sources. Glucose is particularly preferred as a carbon source for cultivating the production strains.

Suitable nitrogen sources include those that can be used by the production strain for formation of biomass. These include ammonia, in gaseous form or in aqueous solution as NH₄OH, or else salts thereof such as, for example, ammonium sulfate, ammonium chloride, ammonium phosphate, ammonium acetate or ammonium nitrate. Additional suitable nitrogen sources include known nitrate salts such as, for example, KNO₃, NaNO₃, ammonium nitrate, Ca(NO₃)₂, Mg(NO₃)₂ and other nitrogen sources such as, for example, urea. The nitrogen sources also include complex mixtures of amino acids such as, for example, yeast extract, proteose peptone, malt extract, soy peptone, casamino acids, corn steep liquor (liquid or else dried as so-called CSD) and also NZ Amine and yeast nitrogen base.

The metered addition of a sulfur source, either as a one-time addition in batch form or as a continuous feed, is required for the efficient production of cysteine and cysteine derivatives. Continuous metered addition of a sulfur source can be achieved by adding the source as a pure feed solution or else by adding it in a mixture with a further feed component such as, for example, glucose.

Suitable sulfur sources are salts of sulfates, sulfites, dithionites, thiosulfates or sulfides. Using the respective acids at a given stability is also contemplated.

Preferred sulfur sources are salts of sulfates, sulfites, thiosulfates and sulfides.

Particularly preferred sulfur sources are salts of sulfates and thiosulfates.

Thiosulfate salts, such as, for example, sodium thiosulfate and ammonium thiosulfate are particularly preferred.

Cultivation can be carried out in so-called batch mode, comprising inoculation of the cultivation medium with a starter culture of the production strain and then cell growth without further feeding of nutrient sources.

Cultivation can also be carried out in so-called fed-batch mode, comprising additional feeding of nutrient sources (feed) after an initial phase of growth in batch mode in order to compensate for the consumption thereof. The feed can consist of the carbon source, the nitrogen source, the sulfur source, one or more vitamins or trace elements important for production, or a combination of the foregoing. The feed components can be metered in together as a mixture or else added separately in individual feed sections. In addition, other media constituents can also be added to the feed, as can additives which specifically increase cysteine production. The feed can be supplied continuously or in portions (discontinuously), or else in a combination of continuous and discontinuous feed. Preference is given to cultivation in fed-batch mode.

Preferred carbon sources in the feed are glucose, sucrose, and glucose- or sucrose-containing vegetable hydrolysates, and mixtures of the preferred carbon sources in any mixing ratio. Glucose is particularly preferred as a feed carbon source.

In preferred embodiments, the carbon source of the culture is metered in such that the content of the carbon source in the fermenter during the production phase does not exceed about 10 g/L, or preferably about 2 g/L, or more preferably about 0.5 g/L, or yet even more preferably about 0.1 g/L. For example, during the production phase, the carbon source content in the fermenter may be about 0.1 g/L, or about 0.2 g/L, or about 0.3 g/L, or about 0.4 g/L, or about 0.5 20 g/L, or about 0.6 g/L, or about 0.7 g/L, or about 0.8 g/L, or about 0.9 g/L, or about 1.0 g/L or about 1.5 g/L, or about 2.0 g/L, or about 2.5 g/L, or about 3.0 g/L, or about 3.5 g/L, or about 3.5 g/L, or about 4.0 g/L, or about 4.5 g/L, or about 5.0 g/L, or about 5.5 g/L, or about 6.0 g/L, or about 6.5 g/L, or about 7.0 g/L, or about 7.5 g/L, or about 8.0 g/L, or about 8.5 g/L, or about 9.0 g/L, or about 9.5 g/L, or about 10.0 g/L.

Suitable nitrogen sources in the feed are preferably ammonia, in gaseous form or in aqueous solution as NH₄OH, and its salts ammonium sulfate, ammonium phosphate, ammonium acetate and ammonium chloride, and additionally urea, KNO₃, NaNO₃ and ammonium nitrate, yeast extract, proteose peptone, malt extract, soy peptone, casamino acids, corn steep liquor and also NZ Amine and yeast nitrogen base. In particular, ammonia or ammonium salts, urea, yeast extract, soy peptone, malt extract or corn steep liquor (in liquid or in dried form) are preferable.

Suitable sulfur sources in the feed are preferably salts of sulfates, sulfites, thiosulfates and sulfides, particularly, sulfur sources in the feed are salts of sulfates and thiosulfates, and more particularly, thiosulfate salts, such as, for example, sodium thiosulfate and ammonium thiosulfate.

As further media additives, salts of the elements phosphorus, chlorine, sodium, magnesium, nitrogen, potassium, calcium, iron and, in trace amounts (i.e., in μM concentrations), salts of the elements molybdenum, boron, cobalt, manganese, zinc, copper and nickel may be added. Furthermore, organic acids (e.g., acetate, citrate), amino acids (e.g., isoleucine) and vitamins (e.g., vitamin Bl, vitamin B6) may be added to the medium.

Cultivation may be carried out under pH and temperature conditions which promote growth and cysteine production of the production strain. Suitable pH values range from about 5 to about 9, or from about 5.5 to about 8, or from about 6.0 to about 7.5. For example, suitable pH values for promoting growth and cysteine production in the production strain is about 5.0, or about 5.1, or about 5.2, or about 5.3, or about 5.4, or about 5.5, or about 6.0, or about 6.5, or about 7.0, or about 7.5, or about 8.0, or about 8.5, or about 9.0.

In at least some preferred embodiments, the temperature range for growth of the production strain is from about 20° C. to about 40° C., or preferably about 25° C. to 37° C., or more preferably from about 28° C. to 34° C. For example, a suitable temperature for growth of the production strain may be about 20° C., or about 21° C., or about 22° C., or about 23° C., or about 24° C., or about 25° C., or about 26° C., or about 27° C., or about 28° C., or about 29° C., or about 30° C., or about 31° C., or about 32° C., or about 33° C., or about 34° C.

Growth of the production strain may optionally occur without an oxygen supply (anaerobic cultivation), or alternatively with oxygen (aerobic cultivation). Preference is given to aerobic cultivation with oxygen.

In the case of aerobic cultivation of the strain according to the invention for cysteine production, an oxygen saturation of at least about 10% (v/v), or preferably at least about 20% (v/v), or more preferably at least about 30% (v/v) is set for the oxygen content. For example, the oxygen content of the media during aerobic cultivation of the strain for cysteine production may be at least about 10% (v/v), or at least about 15%, or at least about 20%, or at least about 25%, or at least about 30%, or at least about 35%, or at least about 40%, or at least about 50%, or at least about 55%, or at least about 60%, or at least about 65%, or at least about 70%, or at least about 75%, or at least about 80%, or at least about 85%, or at least about 90%, or at least about 95% (v/v), or more. In accordance with the prior art, the oxygen saturation in the culture is regulated automatically via a combination of gas supply and stirring speed.

The supply of oxygen is ensured by introduction of compressed air or pure oxygen. Preference is given to aerobic cultivation by introduction of compressed air. The useful range of the compressed air supply in aerobic cultivation is 0.05 vvm to 10 vvm (vvm: introduction of compressed air into the fermentation batch specified in liters of compressed air per liter of fermentation volume per minute). For example, the compressed air supply may be about 0.05, or about 0.1, or about 0.2, or about 0.3, or about 0.4, or about 0.5, or about 1.0, or about 1.5, or about 2.0, or about 2.5, or about 3.0, or about 3.5, or about 4.0, or about 4.5, or about 5.0, or about 5.5, or about 6.0, or about 6.5, or about 7.0, or about 7.5, or about 8.0, or about 8.5, or about 9.0, or about 9.5, or about 10.0 vvm. Preferably, compressed air is introduced in an amount from about 0.2 vvm to 8 vvm, or from about 0.4 to 6 vvm, or from about 0.8 to 5 vvm.

The maximum stirring speed may be about 2500 rpm, or about 2000 rpm, or about 1800 rpm.

In at least some preferred embodiments, the cultivation time may be between about 10 h and about 200 h, or preferably between about 20 h and 120 h, or more preferably between 30 h and 100 h.

Cultivation batches obtained by the method described above contain the cysteine either in dissolved form in the culture supernatant or, oxidized as cystine, in precipitated form. The cysteine or cystine contained in the cultivation batches can either be further used directly without further work-up or else be isolated from the cultivation batch.

In preferred embodiments, the cysteine formed during the process may be isolated. Process steps known per se are available for isolating the cysteine and cystine, including centrifugation, decantation, dissolution of the crude product with a mineral acid, filtration, extraction, chromatography or crystallization, or precipitation. Said process steps can be combined in any form in order to isolate the cysteine in the desired purity. The desired degree of purity depends on downstream uses.

The cystine obtained by work-up can be reduced to cysteine for further use. A process for reducing L-cystine to L-cysteine in an electrochemical process is disclosed in EP 0 235 908.

Various analytical methods for identifying, quantifying and determining the degree of purity of the cysteine or cystine product are available, including spectrophotometry, NMR, gas chromatography, HPLC, mass spectroscopy, gravimetry or else a combination of these analytical methods.

The invention can also be used to produce improved microorganism strains for fermentative production of compounds, the biosynthesis of which starts from 3-phosphoglycerate and leads to L-cysteine and L-cystine via L-serine. This also encompasses microorganism strains for fermentative production of derivatives of L-serine and L-cysteine, including phosphoserine, 0-acetylserine, N-acetylserine and thiazolidine, a condensation product of L-cysteine and pyruvate.

### BRIEF DESCRIPTION OF THE FIGURES

The figures show the plasmids used in the examples.

FIG. 1 shows the 3.4 kb vector pKD13 used in Example 1 and Example 2.

FIG. 2 shows the 6.3 kb vector pKD46 used in Example 1 and Example 3.

FIG. 3 shows the 5 kb vector pKan-SacB used in Example 3.

FIG. 4 shows the 4.2 kb vector pACYC184 used in Example 4.

The invention will be further illustrated by the following examples without being restricted by them:

### Example 1: Production of a ppsA Deletion Mutant in Escherichia coli

The parent strain used for gene isolation and for strain development was Escherichia coli K12 W3110 (commercially available under the strain number DSM 5911 from the DSMZ-German Collection of Microorganisms and Cell Cultures GmbH).

The target of gene inactivation was the coding sequence of the ppsA gene from E. coli. The DNA sequence of the ppsA gene from E. coli K12 (Genbank GeneID: 946209) is disclosed in SEQ ID NO: 1. Nucleotides 333-2711 (identified by E. coli ppsA) encode a phosphoenolpyruvate synthase protein having the amino acid sequence disclosed in SEQ ID NO: 2 (E. coli PpsA).

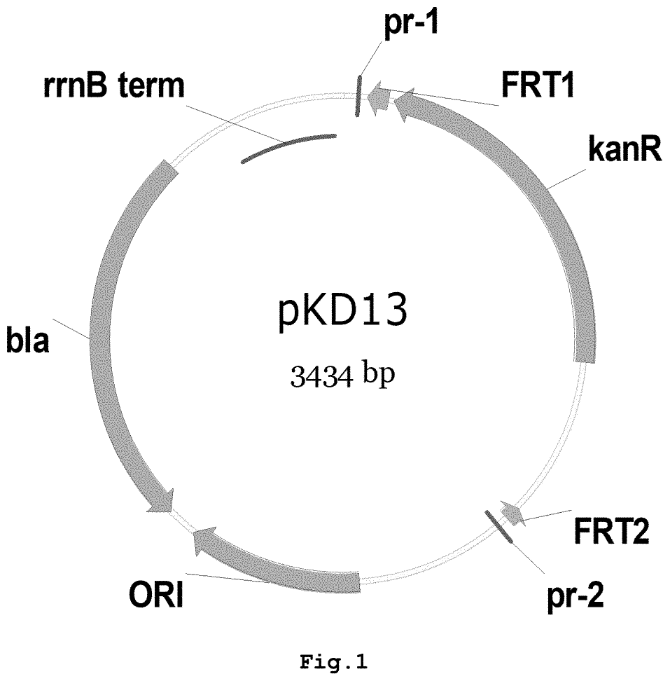

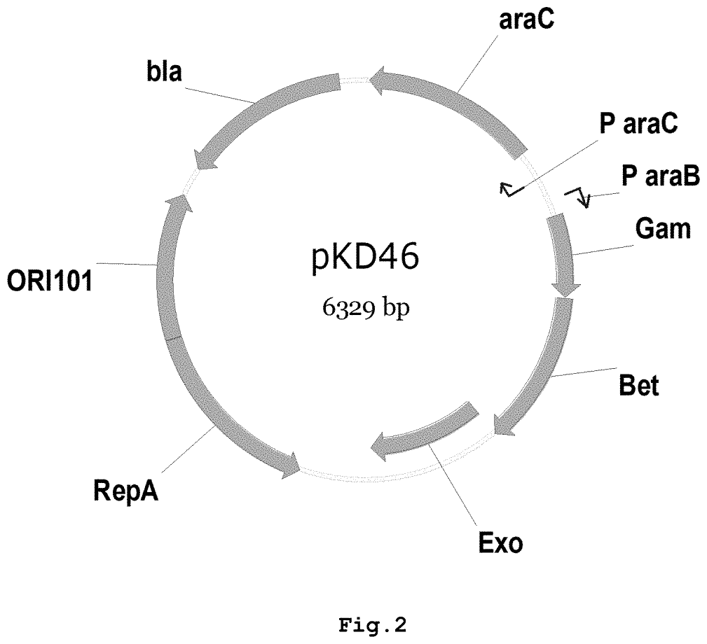

The E. coli ppsA gene was inactivated using Red/ET technology from Gene Bridges GmbH as detailed below (described in the user manual of the “Quick and Easy E. coli Gene Deletion Kit”, see “Technical Protocol, Quick & Easy E. coli Gene Deletion Kit, by Red®/ET® Recombination, Cat. No. K006, Version 2.3, June 2012” and the references cited therein, e.g., Datsenko and Wanner, Proc. Natl. Acad. Sci. USA 97 (2000): 6640-6645). To this end, the following plasmids were used: pKD13, pKD46 and pCP20:
The 3.4 kb plasmid pKD13 (FIG. 1) is disclosed in the “GenBank” gene database under the accession number AY048744.1.
The 6.3 kb plasmid pKD46 (FIG. 2) is disclosed in the “GenBank” gene database under the accession number AY048746.1.
The 9.4 kb plasmid pCP20 is disclosed in Cherepanov and Wackernagel, Gene 158 (1995): 9-14.

To inactivate the ppsA gene in E. coli W3110 by homologous recombination using the Lambda Red system, the following steps were carried out:
1. E. coli W3110 was transformed with the plasmid pKD46 (so-called “Red Recombinase” plasmid, FIG. 2) and an ampicillin-resistant clone was isolated (referred to as W3110×pKD46).
2. A ppsA-specific DNA fragment suitable for inactivation thereof was produced in a PCR reaction (“Phusion™ High-Fidelity” DNA Polymerase, Thermo Scientific™) with DNA of the plasmid pKD13 (FIG. 1) and the primers pps-5f (SEQ ID NO: 7) and pps-6r (SEQ ID NO: 8).
   Primer pps-5f contained 30 nucleotides (nt) from the 5′ region of the ppsA gene (nt 333-362 in SEQ ID NO: 1) and, connected thereto, 20 nt specific for the plasmid pKD13 (referred to as “pr-1” in FIG. 1).
   Primer pps-6r contained 30 nt from the 3′ region of the ppsA gene (nt 2682-2711 in SEQ ID NO: 1, in reverse-complementary form) and, connected thereto, 20 nt specific for the plasmid pKD13 (referred to as “pr-2” in FIG. 1). DNA of the plasmid pKD13 was used to produce, using the primers pps-5f and pps-6r, a 1.4 kb PCR product which contained at both the 5′ end and the 3′ end a 30 nt section of DNA that was specific for the ppsA gene from E. coli W3110. Furthermore, the PCR product contained the expression cassette of the kanamycin resistance gene contained in pKD13 and, flanking the 5′ and 3′ ends of the kanamycin expression cassette, so-called “FRT direct repeats” (referred to as “FRT1” and “FRT2” in FIG. 1), short sections of DNA that were used as a recognition sequence for “FLP recombinase” (contained on the plasmid pCP20) in a later working step for removal of the antibiotic marker kanamycin.
3. The 1.4 kb PCR product was isolated and treated with the restriction endonuclease Dpn I, which is familiar to a person skilled in the art and which only cuts methylated DNA, in order to remove residual pKD13 plasmid DNA. Nonmethylated DNA from the PCR reaction is not degraded.
4. The 1.4 kb PCR product, which is specific for the ppsA gene and contains an expression cassette for the kanamycin resistance gene, was transformed into E. coli W3110×pKD46 and kanamycin-resistant clones were isolated on LBkan plates at 30° C. LBkan plates contained LB medium (10 g/L tryptone, 5 g/L yeast extract, 5 g/L NaCl), 1.5% agar and 15 mg/L kanamycin.
5. Ten of the kanamycin-resistant clones obtained were purified on LBkan plates (i.e., isolation of a clone by singularization) and checked in a PCR reaction to determine whether the kanamycin-resistance cassette had been correctly integrated in the ppsA gene. The genomic DNA used for the PCR reaction (“Phusion™ High-Fidelity” DNA Polymerase, Thermo Scientific™) was isolated using a DNA isolation kit (Qiagen) from cells from the cultivation of kanamycin-resistant clones of E. coli W3110 in Lbkan medium (10 g/L tryptone, 5 g/L yeast extract, 5 g/L NaCl, 15 mg/L kanamycin). Genomic DNA of the E. coli W3110 wild-type strain was used as control. The primers used for the PCR reaction were pps-7f (SEQ ID NO: 9) and pps-8r (SEQ ID NO: 10). Primer pps-7f contained nt 167-188 from SEQ ID NO: 1, and primer pps-8r contained nt 2779-2800 from SEQ ID NO: 1 in reverse-complementary form.
   E. coli W3110 wild-type DNA yielded a DNA fragment of 2630 bp in the PCR reaction, as expected for the intact gene. By contrast, a kanamycin-resistant clone under study yielded a DNA fragment of approx. 1660 bp in the PCR reaction, as expected if the 1.4 kb PCR product had been integrated in the ppsA gene at the sites defined by the primers pps-5f and pps-6r. This result showed that the kanamycin resistance gene had been successfully integrated at the locus of the ppsA gene and that the ppsA gene had thus been inactivated. The clone containing an inactivated ppsA gene was selected and designated W3110-ΔppsA::kan.
6. To eliminate the kanamycin selection marker, W3110-ΔppsA::kan was transformed with the plasmid pCP20 and transformants were selected at 30° C. The 9.4 kb vector pCP20 is disclosed in Cherepanov and Wackernagel (1995), Gene 158: 9-14. Present on the vector pCP20 is the gene of FLP recombinase. FLP recombinase recognizes the FRT sequences flanking the expression cassette of the kanamycin resistance gene and brings about the removal of the kanamycin expression cassette. To this end, the clones obtained at 30° C. were incubated at 37° C. Under these conditions, the expression of FLP recombinase was induced and the replication of the pCP20 vector was suppressed.
   This step produced clones in which the ppsA gene had been inactivated and which had regained sensitivity to kanamycin (so-called “curing” of the antibiotic selection marker). The removal of the kanamycin cassette from the genome of the ΔppsA mutants allows the introduction of further mutations in order to produce double or multiple mutants. W3110-ΔppsA::kan regained kanamycin sensitivity after the treatment with the pCP20 plasmid, which was checked as follows:
   by plating on LB and LBkan plates:
   Growth on LB plates was positive, whereas growth was no longer observed on LBkan plates, which indicated the successful removal of the kanamycin cassette from the genome.
   by PCR reaction:
   To this end, genomic DNA was isolated from the kanamycin-sensitive clones (Qiagen DNA isolation kit) and used in a PCR reaction (“Phusion™ High-Fidelity” DNA Polymerase, Thermo Scientific™) using the primers pps-7f (SEQ ID NO: 9) and pps-8r (SEQ ID NO: 10). E. coli W3110 wild-type DNA yielded a DNA fragment of approx. 2630 bp in the PCR reaction, as expected for the intact gene. By contrast, the kanamycin-sensitive clone yielded a DNA fragment of approx. 300 bp in the PCR reaction, which corresponded to the expected size of the 5′ and 3′ fragments of the inactivated ppsA gene remaining after homologous recombination.
   The strain isolated from this step was designated E. coli W3110-ΔppsA. This strain is distinguished by the fact that it contained an inactivated ppsA gene and that said strain regained sensitivity to the antibiotic kanamycin.

### Example 2: Production of a ppsA Deletion Mutant in Pantoea ananatis

The parent strain used for gene isolation and for strain development was Pantoea ananatis (commercially available under the strain number DSM 30070 from the DSMZ-German Collection of Microorganisms and Cell Cultures GmbH).

The target of gene inactivation was the ppsA gene from Pantoea ananatis. The DNA sequence of the ppsA gene from P. ananatis (Genbank GeneID: 31510655) is disclosed in SEQ ID NO: 3. Nucleotides 417-2801 (identified by P. ananatis ppsA) encode a phosphoenolpyruvate synthase protein having the amino acid sequence disclosed in SEQ ID NO: 4 (P. ananatis PpsA).

The P. ananatis ppsA gene was inactivated using Red®/ET® technology from Gene Bridges GmbH as detailed below (described in the user manual of the “Quick and Easy E. coli Gene Deletion Kit”, see “Technical Protocol, Quick & Easy E. coli Gene Deletion Kit, by Red®/ET® Recombination, Cat. No. K006, Version 2.3, June 2012” and the references cited therein, e.g., Datsenko and Wanner, Proc. Natl. Acad. Sci. USA 97 (2000): 6640-6645). To this end, use was made of the plasmids pKD13 and pRedET.
The 3.4 kb plasmid pKD13 (FIG. 1) is disclosed in the “GenBank” gene database under the accession number AY048744.1.
The commercially available 9.3 kb plasmid pRedET is disclosed in the user manual of the “Quick and Easy E. coli Gene Deletion Kit”, see “Technical Protocol, Quick & Easy E. coli Gene Deletion Kit, by Red®/ET Recombination, Cat. No. K006, Version 2.3, June 2012.”

To inactivate the ppsA gene in P. ananatis by homologous recombination using the Lambda Red system, the following steps were carried out:
1. P. ananatis was transformed with the plasmid pRedET (so-called “Red Recombinase” plasmid) and a tetracycline-resistant clone was isolated (referred to as P. ananatis×pRedET).
2. A ppsA-specific DNA fragment suitable for inactivation thereof was produced in a PCR reaction (“Phusion™ High-Fidelity” DNA Polymerase, Thermo Scientific™) with DNA of the plasmid pKD13 (FIG. 1) and the primers ppsapa-3f (SEQ ID NO: 11) and ppsapa-4r (SEQ ID NO: 12).
   Primer ppsapa-3f contained 49 nt from the 5′ region of the ppsA gene (nt 417-465 in SEQ ID NO: 3) and, connected thereto, 20 nt specific for the plasmid pKD13 (referred to as “pr-1” in FIG. 1).
   Primer ppsapa-4r contained 49 nt from the 3′ region of the ppsA gene (nt 2753-2801 in SEQ ID NO: 3, in reverse-complementary form) and, connected thereto, 20 nt specific for the plasmid pKD13 (referred to as “pr-2” in FIG. 1).
   DNA of the plasmid pKD13 was used to produce, using the primers ppsapa-3f andppsapa4r, a 1.4 kb PCR product which contained at both the 5′ end and the 3′ end a 49 nt section of DNA that was specific for the ppsA gene from P. ananatis. Furthermore, the PCR product contained the expression cassette of the kanamycin resistance gene contained in pKD13 and, flanking the 5′ and 3′ ends of the kanamycin expression cassette, so-called “FRT direct repeats” (referred to as “FRT1” and “FRT2” in FIG. 1), short sections of DNA that allow removal of the antibiotic marker kanamycin in ppsA deletion mutants as required.
3. The 1.4 kb PCR product was isolated and treated with the restriction endonuclease Dpn I, which is familiar to a person skilled in the art and which only cuts methylated DNA, in order to remove residual pKD13 plasmid DNA. Nonmethylated DNA from the PCR reaction is not degraded.
4. The 1.4 kb PCR product, which is specific for the ppsA gene and contains an expression cassette for the kanamycin resistance gene, was transformed into P. ananatis×pRedET and kanamycin-resistant clones were isolated on LBkan plates at 30° C. LBkan plates contained LB medium (10 g/L tryptone, 5 g/L yeast extract, 5 g/L NaCl), 1.5% agar and 15 mg/L kanamycin.
5. A kanamycin-resistant clone was purified on LBkan plates (i.e., isolation of a clone by singularization) and checked in a PCR reaction to determine whether the kanamycin-resistance cassette had been correctly integrated in the ppsA gene.
   The genomic DNA used for the PCR reaction (“Phusion™ High-Fidelity” DNA Polymerase, Thermo Scientific™) was isolated using a DNA isolation kit (Qiagen) from cells from the cultivation of the kanamycin-resistant clone of P. ananatis in Lbkan medium (10 g/L tryptone, 5 g/L yeast extract, 5 g/L NaCl, 15 mg/L kanamycin). Genomic DNA of the P. ananatis wild-type strain was used as control. The primers used for the PCR reaction were ppsapa-1f (SEQ ID NO: 13) and ppsapa-2r (SEQ ID NO: 14). Primer ppsapa-1f contained nt 281-302 in SEQ ID NO: 3, and primer ppsapa-2r contained nt 2901-2922 in SEQ ID NO: 3, in reverse-complementary form.
   P. ananatis wild-type DNA yielded a DNA fragment of 2640 bp in the PCR reaction, as expected for the intact gene. By contrast, a kanamycin-resistant clone under study yielded a DNA fragment of approx. 1670 bp in the PCR reaction, as expected if the 1.4 kb PCR product had been integrated in the ppsA gene at the sites defined by the primers ppsapa-3f (SEQ ID NO: 11) and ppsapa-4r (SEQ ID NO: 12). This result showed that the kanamycin resistance gene had been successfully integrated at the locus of the ppsA gene and that the ppsA gene had thus been inactivated. The clone containing an inactivated ppsA Gen was selected and designated P. ananatis-ΔppsA::kan.

### Example 3: Production of Escherichia coli W3110-ppsA-MHI

E. coli W3110-ppsA-MHI, characterized by mutations of the ppsA structural gene in a manner causing attenuation of enzyme activity, was produced by using the combination, known to a person skilled in the art, of Lambda Red recombination and counter-selection screening for genetic modification (see, for example, Sun et al., Appl. Env. Microbiol. (2008) 74: 4241-4245). The DNA sequence of the gene ppsA-MHI is disclosed in SEQ ID NO: 5 (ppsA-MHI), encoding a protein having the sequence as specified in SEQ ID NO: 6 (PpsA-MHI).

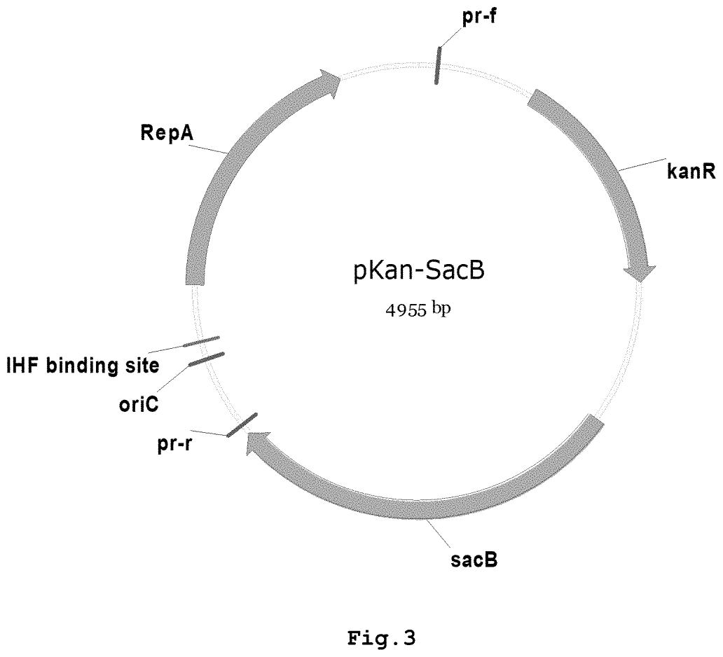

The procedure was as follows:
1. A 2.6 kb DNA fragment comprising parts of the ppsA WT gene (nt 167 to nt 2800 in SEQ ID NO: 1), i.e., the cds and also 5′ and 3′ flanking sequences, was isolated from genomic DNA of E. coli W3110 by PCR using the primers pps-7f (SEQ ID NO: 9) and pps-8r (SEQ ID NO: 10).
2. ppsA-MHI was obtained from the ppsA WT gene by successively introducing the mutations into the ppsA WT gene by “site-directed” mutagenesis. This was done using the commercially available cloning kit “QuickChange II Site-Directed Mutagenesis Kit” from Agilent in accordance with the instructions in the user manual.
3. In order to exchange the ppsA WT gene of E. coli W3110 for ppsA-MHI, the 3.2 kb Kan-sacB cassette was first isolated from the plasmid pKan-SacB (FIG. 3) by PCR using the primers pps-9f (SEQ ID NO: 15) and pps-10r (SEQ ID NO: 16).
   The plasmid pKan-sacB contains expression cassettes for both the kanamycin (Kan) resistance gene and the sacB gene encoding the enzyme levansucrase.
   The primer pps-9f contained 30 nt starting from the start ATG of the ppsA gene (nt 333-362 in SEQ ID NO: 1) and, connected thereto, 20 nt specific for the plasmid pKan-SacB (referred to as “pr-f” in FIG. 3).
   The primer pps-10r contained 30 nt from the stop codon of the ppsA gene (nt 2682-2711 in SEQ ID NO: 1, in reverse-complementary form) and, connected thereto, 21 nt specific for the plasmid pKan-SacB (referred to as “pr-r” in FIG. 3).
4. E. coli W3110×pKD46 (for production thereof, see Example 1) was transformed with the ppsA-specific 3.2 kb PCR product and kanamycin-resistant clones were isolated.
5. The clones were seeded onto LBSC plates (10 g/L tryptone, 5 g/L yeast extract, 7% sucrose, 1.5% agar and 15 mg/L kanamycin).
   Clones containing an integrated sacB gene produced toxic levan from sucrose, and this led to growth inhibition. Such clones were selected and checked in a PCR reaction to determine whether the Kan-sacB cassette had been correctly integrated in the ppsA gene. The genomic DNA used for the PCR reaction (“Phusion™ High-Fidelity” DNA Polymerase, Thermo Scientific™) had been obtained previously using a DNA isolation kit (Qiagen) from cells from the cultivation of kanamycin-resistant clones of E. coli W3110 in Lbkan medium (10 g/L tryptone, 5 g/L yeast extract, 5 g/L NaCl, 15 mg/L kanamycin). Genomic DNA of the E. coli W3110 wild-type strain was used as control. The primers used for the PCR reaction were pps-7f (SEQ ID NO: 9) and pps-8r (SEQ ID NO: 10).
   E. coli W3110 wild-type DNA yielded a DNA fragment of 2630 nt in the PCR reaction, as expected for the intact gene. By contrast, kanamycin-resistant clones yielded a DNA fragment of approx. 3400 nt in the PCR reaction, as expected if the 3.2 kb PCR product had been integrated in the ppsA gene at the sites defined by the primers pps-9f (SEQ ID NO: 15) and pps-10r (SEQ ID NO: 16). This result showed that the Kan-sacB cassette had been successfully integrated at the locus of the ppsA gene and that the ppsA gene had thus been inactivated. A clone containing an integrated Kan-sacB cassette was selected and designated W3110-ΔppsA::kan-sacB×pKD46.
6. In the next step, the Kan-sacB cassette was exchanged for the ppsA-MHI gene. To this end, a 2.5 kb DNA fragment was amplified from the ppsA-MHI DNA fragment from step 2 in a PCR reaction (“Phusion™ High-Fidelity” DNA Polymerase, Thermo Scientific™) using the primers pps-11f (SEQ ID NO: 17) and pps-12r (SEQ ID NO: 18). Primer pps-11f contained nt 300-319 in SEQ ID NO: 1, and primer pps-12r contained nt 2743-2763 in SEQ ID NO: 1, in reverse-complementary form.
7. The 2.5 kb ppsA-MI-II gene was transformed into E. coli W3110-ΔppsA::kan-sacB×pKD46 and clones were selected on LBS plates (10 g/L tryptone, 5 g/L yeast extract, 7% sucrose, 1.5% agar) without kanamycin. Only clones which no longer contained an active sacB gene could grow on LBS plates.
   These clones were seeded onto LBkan plates in order to select those clones which also no longer contained an active Kan gene and the growth of which was inhibited in the presence of kanamycin.
   Clones exhibiting positive growth in the presence of sucrose and negative growth in the presence of kanamycin were selected and checked in a PCR reaction to determine whether the Kan-sacB cassette had been correctly replaced by the ppsA-MHI gene.
   Genomic DNA was obtained using a DNA isolation kit (Qiagen) from cells from the cultivation in LB medium (10 g/L tryptone, 5 g/L yeast extract, 5 g/L NaCl). Genomic DNA of the E. coli W3110 wild-type strain was used as control. The primers used for the PCR reaction were pps-7f (SEQ ID NO: 9) and pps-8r (SEQ ID NO: 10). PCR products of the expected size of 2630 nt were analyzed by DNA sequencing (Eurofins Genomics). Clones containing a correctly integrated ppsA-MI-II gene yielded the DNA sequence as disclosed in SEQ ID NO: 5, encoding a protein corresponding to the sequence from SEQ ID NO: 6. A clone containing a correct ppsA-MI-II gene containing the mutations V126M, R427H and V434I was selected and designated E. coli W3110-ppsA-MHI.

### Example 4: Generation of Cysteine Production Strains

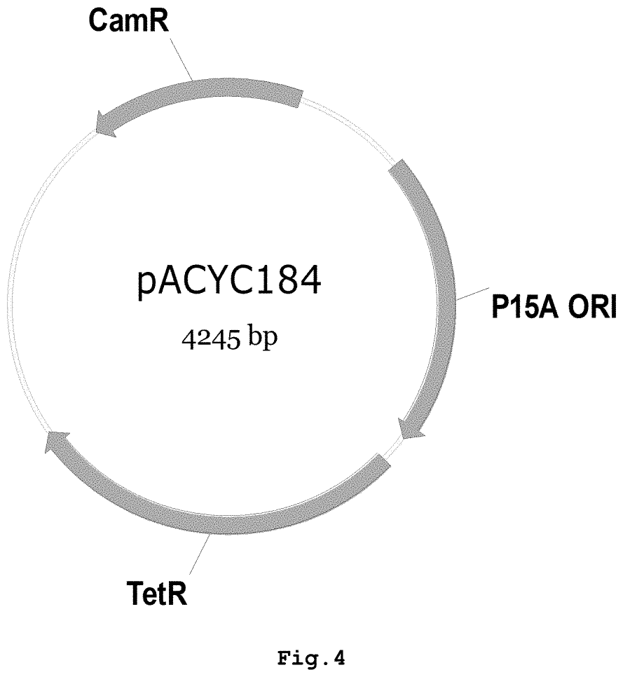

The cysteine-specific production plasmid used was the plasmid pACYC184-cysEX-GAPDH-ORF306-serA317 derived from the parent vector pACYC184 (FIG. 4). pACYC184-cysEX-GAPDH-ORF306-serA317 is a derivative of the plasmid pACYC184-cysEX-GAPDH-ORF306 disclosed in EP 0 885 962 B1. The plasmid pACYC184-cysEX-GAPDH-ORF306 contains not only the origin of replication and a tetracycline resistance gene (parent vector pACYC184), but also the cysEX allele, which encodes a serine 0-acetyltransferase having a reduced feedback inhibition by cysteine, and the efflux gene ydeD (ORF306), the expression of which is controlled by the constitutive GAPDH promoter.

Furthermore, pACYC184-cysEX-GAPDH-ORF306-serA317 additionally contains the serA317 gene fragment, which is cloned after the ydeD (ORF306) efflux gene and which encodes the N-terminal 317 amino acids of the SerA protein (total length: 410 amino acids). The E. coli serA gene is disclosed in the “GenBank” gene database with the gene ID 945258. serA317 is disclosed in Bell et al., Eur. J. Biochem. (2002) 269: 4176-4184, referred to therein as “NSD:317”, and encodes a serine feedback-resistant variant of 3-phosphoglycerate dehydrogenase. The expression of serA317 is controlled by the serA promoter.

The strains E. coli W3110, E. coli W3110-ΔppsA, E. coli W3110-ppsA-MHI, P. ananatis and P. ananatis-ΔppsA::kan were each transformed with the plasmid pACYC184-cysEX-GAPDH-ORF306-serA317 (referred to as pCYS in the following examples). Transformation was carried out according to the prior art by means of electroporation, as described in EP 0 885 962 B1.

Plasmid-bearing transformants were selected on LBtet agar plates (10 g/L tryptone, 5 g/L yeast extract, 5 g/L NaCl, 1.5% agar, 15 mg/L tetracycline). Selected transformants were checked for the transformed pCYS plasmid by plasmid isolation by means of the QIAprep Spin Plasmid Kit (Qiagen) and restriction analysis. Transformants containing a correctly incorporated plasmid pCYS were cultivated to check ppsA enzyme activity (Example 5) and to determine cysteine production (Example 6 and Example 7).

### Example 5: Determination of ppsA Enzyme Activity

What was determined was the ppsA enzyme activity of the E. coli strains W3110, W3110-ΔppsA, W3110-ppsA-MHI, each transformed with the production plasmid pCYS (Example 4). Cells from the shake-flask cultivation of the three strains in 50 ml of SM1 medium (for the composition thereof, see Example 6) were pelleted by centrifugation for 10 min and washed once with 10 ml of 0.9% (w/v) NaCl. The cell pellets were taken up in 10 ml of assay buffer (100 mM Tris-HCl, pH 8.0; 10 mM MgCl₂) and a cell extract was prepared.

The cell homogenizer FastPrep-24™ 5G from MP Biomedicals was used. To this end, 2×1 ml of cell suspension were disrupted in 1.5 ml tubes prefabricated by the manufacturer and containing glass beads (“Lysing Matrix B”) (3×20 sec at a shaking frequency of 6000 rpm with a sec pause each time between the intervals). The resulting homogenate was centrifuged and the supernatant was used as cell extract for determining activity.

The protein content of the extract was determined by means of a Qubit 3.0 Fluorometer from Thermo Fisher Scientific using the “Qubit® Protein Assay Kit” according to the manufacturer's instructions.

To determine ppsA enzyme activity, the phosphate detection kit “Malachite Green Phosphate Assay Kit” from Sigma Aldrich (catalog number MAK307) was used in accordance with the manufacturer's instructions. The basis thereof is the conversion of pyruvate with ATP to form phosphoenolpyruvate in equilibrium reaction (4) by ppsA enzyme activity. This produces stoichiometric amounts of phosphate, which is used for determining activity.
The assays contained 100 μg of cell extract, 4 mM Na pyruvate and 4 mM ATP in 1 ml of assay buffer (100 mM Tris-HCl, pH 8.0; 10 mM MgCl₂).
The various assays were incubated at 30° C.
0 min, 10 min, 20 min, 30 min and 60 min after the start of incubation, 50 μl of the respective assay were removed, added to 750 μl of H₂O, and lastly admixed with 200 μl of reagent from the “Malachite Green Phosphate Assay Kit”.
After 30 min of incubation, the amount of phosphate formed was determined photometrically by determination of the absorbance at 620 nm, with the aid of a phosphate standard curve and according to the manufacturer's instructions. Lastly, ppsA enzyme activity in U/ml extract (1 U=μmol substrate turnover/min) was determined from the measured amount of phosphate, based on the time of sampling from the respective assay. Specific ppsA enzyme activity was calculated by basing the ppsA enzyme activity on 1 mg of total protein of the cell extract (U/mg protein).

TABLE 1
Determination of ppsA enzyme activity

| | Specific ppsA | Relative enzyme activity (in |
|---|---|---|
| Strain | activity | relation to W3110 × pCys) |
| W3110-ΔppsA × pCYS | 0.00 U/mg | 0% |
| W3110-ppsA-MHI × pCYS | 0.42 U/mg | 26.8% |
| W3110 × pCYS | 1.58 U/mg | 100% |

### Example 6: Cysteine Production in a Shake Flask

As a preculture for cultivation in a shake flask, 3 ml of LB medium (10 g/L tryptone, 5 g/L yeast extract, 10 g/L NaCl) which additionally contained 15 mg/L tetracycline were inoculated with the respective strain and incubated in a shaker at 30° C. and 135 rpm for 16 h. The strains studied were E. coli W3110, W3110-ΔppsA, W3110-ppsA-MHI, and, in a second experiment, P. ananatis and P. ananatis-ΔppsA::kan, each transformed with the production plasmid pCYS (Example 4).

Main culture: Thereafter, a portion of the respective preculture was transferred to a 300 ml Erlenmeyer flask (baffled) containing 30 ml of SM1 medium containing 15 g/L glucose, 5 mg/L vitamin B1 and 15 mg/L tetracycline.

Composition of the SM1 medium: 12 g/L K2HPO₄, 3 g/L KH₂PO₄, 5 g/L (NH₄)₂SO₄, 0.3 g/L MgSO₄×7 H₂O, 0.015 g/L CaCl₂×2 H₂O, 0.002 g/L FeSO₄×7 H₂O, 1 g/L Na₃ citrate×2 H₂O, 0.1 g/L NaCl; 1 ml/L trace element solution.

Composition of the trace element solution: 0.15 g/L Na₂MoO₄×2 H₂O, 2.5 g/L H₃BO₃, 0.7 g/L CoCl₂×6 H₂O, 0.25 g/L CuSO₄×5 H₂O, 1.6 g/L MnCl₂×4 H₂O, 0.3 g/L ZnSO₄×7 H₂O.

The main culture was inoculated with enough preculture to establish an initial cell density OD₆₀₀/ml (optical density of the main culture, measured at 600 nm) of 0.025/ml. Starting from this, the entire 30 ml batch was incubated at 30° C. and 135 rpm for 24 h.

After 24 h, samples were taken and the cell density OD₆₀₀/ml and the total cysteine content in the culture supernatant were determined, the colorimetric assay by Gaitonde (Gaitonde, M. K. (1967), Biochem. J. 104, 627-633) being used for quantitative determination of cysteine. It should be borne in mind that, under the highly acidic reaction conditions, this assay does not distinguish between cysteine and the condensation product of cysteine and pyruvate, 2-methylthiazolidine-2,4-dicarboxylic acid (thiazolidine), that is described in EP 0 885 962 B1. L-cystine, which is formed by oxidation of two cysteine molecules according to equation (2), is likewise detected as cysteine in the assay by reduction with dithiothreitol in dilute solution at pH 8.0. The results are reported in Table 2 for the E. coli strains mentioned and in Table 3 for the P. ananatis strains.

TABLE 2
Cell density and total cysteine content after
a culture time of 24 h in a shake flask

| | Cell density | Cysteine |
|---|---|---|
| Strain | OD₆₀₀/ml | (g/L) |
| W3110 | 7.0 | 0.00 |
| W3110 × pCYS | 3.4 | 0.46 |
| W3110-ppsA-MHI × pCYS | 4.8 | 0.73 |
| W3110-ΔppsA × pCYS | 5.2 | 0.72 |

TABLE 3
Cell density and total cysteine content after
a culture time of 24 h in a shake flask

| | Cell density | Cysteine |
|---|---|---|
| Strain | OD₆₀₀/ml | (g/L) |
| P. ananatis × pCYS | 2.5 | 0.09 |
| P. ananatis-ΔppsA::kan × pCYS | 2.4 | 0.31 |

### Example 7: Cysteine Production in a Fermenter

A comparison was made between E. coli W3110×pCYS, W3110-ppsA-MHI×pCYS and W3110-ΔppsA×pCYS in production-scale fed-batch fermentation.

Preculture 1:

20 ml of LB medium containing 15 mg/L tetracycline were inoculated with the respective strain in a 100 ml Erlenmeyer flask and incubated on a shaker (150 rpm, 30° C.) for 7 h.

Preculture 2:

Thereafter, the entire preculture 1 was transferred to 100 ml of SM1 medium supplemented with 5 g/L glucose, 5 mg/L vitamin B1 and 15 mg/L tetracycline (for the composition of SM1 medium, see Example 6).

The cultures were shaken in Erlenmeyer flasks (1 L volume) at 30° C. for 17 h at 150 rpm (Infors incubator shaker). Following this incubation, the cell density OD₆₀₀/ml was between 3 and 5.

Main Culture:

Fermentation was carried out in a “DASGIP® Parallel Bioreactor System for Microbiology” fermenter from Eppendorf. Culture vessels with a total volume of 1.8 L were used. The fermentation medium (900 ml) contained 15 g/L glucose, 10 g/L tryptone (Difco), 5 g/L yeast extract (Difco), 5 g/L (NH₄)₂SO₄, 1.5 g/L KH₂PO₄, 0.5 g/L NaCl, 0.3 g/L MgSO₄×7 H₂O, 0.015 g/L CaCl₂)×2 H₂O, 0.075 g/L FeSO₄×7 H₂O, 1 g/L Na₃ citrate×2 H₂O and 1 ml of trace element solution (see Example 6), 0.005 g/L vitamin Bl and 15 mg/L tetracycline.

The pH in the fermenter was initially adjusted to 6.5 by pumping in a 25% NH₄OH solution. During the fermentation, the pH was maintained at a value of 6.5 by automatic correction with 25% NH₄OH. For inoculation, 100 ml of preculture 2 were pumped into the fermenter vessel. The initial volume was therefore about 1 L. The cultures were initially stirred at 400 rpm and aerated with compressed air sterilized via a sterile filter at an aeration rate of 2 vvm (volume of air per volume of culture medium per minute). Under these starting conditions, the oxygen probe was calibrated to 100% saturation prior to inoculation.

The target value for the O₂ saturation during the fermentation was set to 30%. After the O₂ saturation had fallen below the target value, a regulation cascade was started in order to bring the O₂ saturation back up to the target value. This involved first increasing the gas supply continuously (to a maximum of 5 vvm) and then increasing the stirring speed continuously (to a maximum of 1500 rpm).

The fermentation was carried out at a temperature of 30° C. After a fermentation time of 2 h, a sulfur source in the form of a sterile 60% (w/v) stock solution of sodium thiosulfate×5 H₂O was fed in at a rate of 1.5 ml per hour.

Once the glucose content in the fermenter had fallen from an initial 15 g/L to approx. 2 g/L, a 56% (w/w) glucose solution was continuously metered in. The feeding rate was adjusted such that the glucose concentration in the fermenter no longer exceeded 2 g/L from then on. Glucose was determined using a glucose analyzer from YSI (Yellow Springs, Ohio, USA).

The fermentation time was 48 h. Thereafter, samples were taken from the fermentation batch and separate determination of the content of L-cysteine and the derivatives derived therefrom in the culture supernatant (primarily L-cysteine and thiazolidine) and in the precipitate (L-cystine) was carried out. For this purpose, use was made of the colorimetric assay by Gaitonde in each case (Gaitonde, M. K. (1967), Biochem. J. 104, 627-633). The L-cystine present in the precipitate first had to be dissolved in 8% (v/v) hydrochloric acid before it could be quantified in the same way. Lastly, the total amount of cysteine was determined as the sum total of cysteine in the pellet and in the supernatant.

As summarized in Table 4, the cell density OD₆₀₀/ml of the strains studied was comparable, although somewhat higher for the control strain W3110×pCYS. By contrast, volume production of cysteine (in g/L) was significantly higher both in W3110-ppsA-MHI×pCYS and in W3110-ΔppsA×pCYS (by a factor of approx. 3) than in the control strain W3110×pCYS containing the wild-type ppsA gene.

Under the controlled fermentation conditions, the result therefore achieved for the production scale is that attenuation of activity or inactivation in respect of ppsA enzyme activity leads to significantly improved cysteine production and is therefore a suitable measure for improving strains, which result has not been described previously and is also unexpected for a person skilled in the art on account of the prior art.

TABLE 4
Cell density and total cysteine content after
a culture time of 24 h in a fermenter

| | Cell density | Cysteine |
|---|---|---|
| Strain | OD₆₀₀/ml | (g/L) |
| W3110 × pCYS | 95.6 | 8.7 |
| W3110-ppsA-MHI × pCYS | 85.0 | 26.4 |
| W3110-ΔppsA × pCYS | 85.4 | 25.0 |

### ABBREVIATIONS USED IN THE FIGURES

bla: Gene conferring resistance to ampicillin ((3-lactamase)
rrnB term: rrnB terminator for transcription
kanR: Gene conferring resistance to kanamycin
ORI: Origin of replication
pr-1: Binding site 1 for primer
pr-2: Binding site 2 for primer
FRT1: Recognition sequence 1 for FLP recombinase
FRT2: Recognition sequence 2 for FLP recombinase
araC: araC gene (repressor gene)
P araC: Promoter of the araC gene
P araB: Promoter of the araB gene
Gam: Lambda phage Gam recombination gene
Bet: Lambda phage Bet recombination gene
Exo: Lambda phage Exo recombination gene
ORI101: Temperature-sensitive origin of replication
RepA: Gene for plasmid replication protein A
sacB: Levansucrase gene
pr-f: Binding site f for primer (forward)
pr-r: Binding site r for primer (reverse)
OriC: Origin of replication C
IHF: Binding site for DNA binding protein IHF (“Integration Host Factor”)
CamR: Gene conferring resistance to chloramphenicol
TetR: Gene conferring resistance to tetracycline
P15A ORI: Origin of replication

1.-12. (canceled)

13. A microorganism strain suitable for fermentative production of L-cysteine, comprising a genetically modified microorganism strain having inactivated or reduced enzyme activity relative to the activity of the corresponding wild-type enzyme of the enzyme class identified by the number EC 2.7.9.2 in the KEGG database; and increased L-cysteine production relative to a microorganism strain having wild-type enzyme activity of the enzyme class identified by the number EC 2.7.9.2 in the KEGG database,
wherein the gene encoding said enzyme activity is ppsA.

14. The microorganism strain of claim 13, wherein the strain is from the Enterobacteriaceae or Corynebacteriaceae family.

15. The microorganism strain of claim 13, wherein the microorganism strain is selected from the group consisting of Escherichia coli, Pantoea ananatis and Corynebacterium glutamicum.

16. The microorganism strain of claim 13, wherein the microorganism strain is selected from the group consisting of Escherichia coli and Pantoea ananatis.

17. The microorganism strain of claim 13, wherein the microorganism strain is a strain of the species Escherichia coli.

18. The microorganism strain of claim 14, wherein the genome of the microorganism strain contains at least one mutation in the ppsA gene.

19. The microorganism strain of claim 18, wherein the mutated ppsA gene is selected from the group consisting of the ppsA gene from Escherichia coli, the ppsA gene from Pantoea ananatis, and a gene homologous to these genes, wherein a gene homologous to these genes is a DNA sequence which is at least 80% identical to these genes.

20. The microorganism strain of claim 19, wherein the coding DNA sequence of the ppsA gene is SEQ ID NO: 5.

21. The microorganism strain of claim 19, wherein the strain overexpresses a serine 0-acetyltransferase protein having a reduced feedback inhibition by cysteine; an efflux gene; and a serine feedback-resistant variant of 3-phosphoglycerate dehydrogenase.

22. The microorganism strain of claim 14, wherein the relative enzyme activity of the enzyme class identified by the number EC 2.7.9.2 in the KEGG database is reduced by at least 25% in this strain in relation to the activity of the corresponding wild-type enzyme.

23. The microorganism strain of claim 14, wherein the relative enzyme activity of the enzyme class identified by the number EC 2.7.9.2 in the KEGG database is reduced by at least 70% in this strain in relation to the activity of the corresponding wild-type enzyme.

24. The microorganism strain of claim 14, wherein the strain has no enzyme activity of the enzyme class identified by the number EC 2.7.9.2 in the KEGG database.

25. The microorganism strain of claim 21, wherein the relative enzyme activity of the enzyme class identified by the number EC 2.7.9.2 in the KEGG database is reduced by at least 25% in this strain in relation to the activity of the corresponding wild-type enzyme.

26. The microorganism strain of claim 21, wherein the relative enzyme activity of the enzyme class identified by the number EC 2.7.9.2 in the KEGG database is reduced by at least 70% in this strain in relation to the activity of the corresponding wild-type enzyme.

27. The microorganism strain of claim 21, wherein the strain has no enzyme activity of the enzyme class identified by the number EC 2.7.9.2 in the KEGG database.

28. A fermentative process for producing L-cysteine, comprising:
providing a microorganism strain, selected from the group consisting of Escherichia coli, Pantoea ananatis and Corynebacterium glutamicum and suitable for fermentive production of L-cysteine, wherein the strain comprises inactivated or reduced PpsA enzyme activity relative to the activity of the corresponding wild-type PpsA enzyme, and increased L-cysteine production relative to a microorganism strain having wild-type enzyme activity of the PpsA enzyme;
culturing the microorganism strain under fermentation conditions to produce L-cysteine; and
collecting the cysteine from the culture.

29. The process of claim 31, wherein the microorganism strain further comprises at least one mutation in a ppsA gene, selected from the group consisting of the ppsA gene from Escherichia coli, the ppsA gene from Pantoea ananatis, and a gene homologous to these genes, wherein a gene homologous to these genes is a DNA sequence which is at least 80% identical to these genes.

30. The process of claim 32, wherein the microorganism strain overexpresses a serine O-acetyltransferase protein having a reduced feedback inhibition by cysteine; the efflux gene; and a serine feedback-resistant variant of 3-phosphoglycerate dehydrogenase.
